(19) United States
(12) Patent Application Publication
(10) Pub. No.: US 2002/0031818 A1
(43) Pub. Date: Mar. 14, 2002

(54) Modification of Mdm2 activity

(76) Inventors: Ze?apos;ev A. Ronai, Suffern, NY (US); Serge Y. Fuchs, Goldens Bridge, PA (US)

Correspondence Address: DARBY & DARBY P.C., 805 Third Avenue, New York, NY, 10022, US

(21) Appl. No.: 09/888,077

(22) Filed: Jun. 22, 2001

Related U.S. Application Data
(60) Provisional application No. 60/213,343, filed on Jun. 22, 2000.

(51) Int. Cl. G01N 33/574; A61K 38/48; C12N 9/64

(52) U.S. Cl. 435/226; 424/94.63; 435/7.23; 435/23

(57) ABSTRACT

The present invention discloses a method for modulating Mdm2 activity by altering the level of sumoylation and ubiquitination of the Mdm2 protein, which in turn may modulate p53 activity. The invention further provides methods of detecting sumoylation of Mdm2, an assay system for identifying a test compound that regulates sumoylation of Mdm2, and a method of treating a condition of uncontrolled cell growth.

### PRIORITY

This application claims priority under 35 U.S.C. § 119 from provisional patent application Ser. No. 60/213,343, filed Jun. 22, 2000, which is hereby incorporated by reference in its entirety.

The research leading to the present invention was supported by National Institutes of Health Grants CA55908 and GM55059. The United States Government may have certain rights in this invention.

### BACKGROUND OF THE INVENTION

Proteolysis plays an important role in regulating the cell's proliferation, differentiation and response to stress. A delicate balance between protein protection from—or targeting for—degradation underlies the regulation of proteolysis and determines the duration and magnitude of activities elicited by key regulatory proteins. Covalent attachment of polyubiquitin is required for the efficient degradation of proteins by the 26S proteasome complex (Hershko and Ciechanover, Annu. Rev. Biochem. 67:425-429, 1998). Conjugation of the ubiquitin polypeptide is mediated by multiple enzymatic reactions catalyzed by a single ubiquitin-activating enzyme (E 1), a few ubiquitin-conjugating enzymes (E2s) and a large variety of ubiquitin ligases (E3s). Whereas recognition of substrates by E3 ubiquitin ligases dictates the specificity of protein ubiquitination, the intrinsic ubiquitin ligase activity represents a rate-limiting step of ubiquitin conjugation. Therefore, the mechanisms underlying the regulation of E3 are central to the control of proteasome-dependent protein degradation in vivo.

Two major classes of E3 ubiquitin ligases are known. The first is represented by HECT (homologous to E6AP carboxy-terminus) ligases, in which the formation of ubiquitin-thioester intermediates followed by a direct transfer of ubiquitin onto a substrate (Nuber and Scheffner, J. Biol. Chem. 274:7576-7582,1999) as shown for E6AP, RSP5, and Nedd4 (Huibregtse et al., Proc Natl Acad Sci U S A 92:2563-2567, 1995; Schwarz et al., J. Biol. Chem. 273:12148-12154, 1998). The other E3 class, whose activity depends on the presence of a RING finger motif, does not form thioester intermediates with ubiquitin (Chen et al., J Biol Chem. In Press). Small RING finger protein ROC1/Rbx1/Hrt1 mediates ubiquitin ligation within the multi-protein E3 complexes, such as Skp1-Cullin1-F-box protein-ROC1 (SCF-ROC1) and VHL-elongin B-elongin C-Cullin2-Roc1 (VCB-Cul2-ROC1) (Kamura et al., Genes Dev. 13:2928-2933,1999; Tan et al, Mol. Cell. 3:527-533,1999; Wu et al., Mol. Cell. Biol. 20:1382-1393, 2000). These E3s mediate ubiquitination of cyclin-dependent kinase inhibitors, yeast cyclins, inhibitor of NF-kB transcription factor (IkB), β-catenin and a subunit of hypoxia-inducible factor (reviewed in Deshaies, Cell. Dev. Biol. 15:435-467,1999). APC11, αROC₁ homologue, participates in the formation of the anaphase-promoting complex (APC) ubiquitin ligase and mediates ubiquitination of cyclins A and B, Pds 1 and Geminin (reviewed in Zachariae and Nasmyth, Genes Dev. 13:2039-2058, 1999).

Whereas ROC1 and APC11 are part of a multi-protein complex, and rely on their partners (i.e., F box proteins) for substrate recognition, recent studies reveal a rapidly growing number of RING finger proteins that directly bind their substrate and exhibit ubiquitin ligase activity. Among those are mammalian homologues of seven in absentia, AO7, cCbl/Sli-1, BRCA1, and Mdm2 (Hu et al., Genes Dev. 11:2701-2714, 1997; Honda et al., EMBO J. 18:22-27, 1999; Levkowitz et al., Mol Cell.4:1029-1240,1999; Lorick et al., Proc. Natl. Acad. Sci. USA 96:11364-11369, 1999).

As an oncogene, Mdm2's transforming potential is largely attributed to its ability to down regulate the functions of the p53 tumor suppressor protein. The disruption of p53 biological activities often seen in human tumors is, at least in part, attributed to Mdm2 over-expression. Association of Mdm2 with p53 abrogates p53 transcriptional activities (Chen et al., Mol. Med. 1: 142-152, 1995). Moreover, Mdm2 has been implicated in the proteasome-dependent degradation of p53 (Haupt et al., Nature 387:296-299, 1997; Kubbutat et al., Nature 387:299-303, 1997). Mdm2 has been shown to target p53 ubiquitination in vitro and in vivo (Honda et al., FEBS Lett. 420:25-27, 1997; Fuchs et al., Oncogene 17:2543-2547, 1998). Although initially believed to form the ubiquitin-thioester intermediate (Honda et al., FEBS Lett. 420:25-27,1997), Mdm2 does not contain the HECT domain. Recent evidence suggests that Mdm2 mediates p53 ubiquitination via the carboxyl terminal RING finger domain and that Mdm2 is a bona fide RING finger E3 ubiquitin ligase (Fang et al., J Biol Chem 275, 8945-51:2000; Honda and Yasuda, Oncogene 19:1473-6, 2000).

The ubiquitination of p53 by Mdm2 is tightly regulated in mammalian cells. Mdm2 effects on p53 are attributed to the abundance of Mdm2 expression, the levels of Mdm2-p53 association, and modulation of Mdm2 ubiquitin ligase activity. Mdm2 levels are regulated by its substrate p53, which activates Mdm2 gene transcription (Barak et al., EMBO J 2:461-468, 1993). Stabilization of p53 in response to stress and DNA damage is due to Mdm2 dissociation from phosphorylated p53 (Shieh et al., Cell 91:325-334, 1997; Siliciano et al., Genes Dev. 11:3471-3481, 1997; Fuchs et al., Oncogene 17:2543-2547, 1998). The accumulation of p53 may be also attributed to the inhibition of Mdm2 ubiquitin ligase activity upon its association with p14^{ARF} or MdmX (Kamijo et al., Proc. Natl. Acad. Sci. USA 95:8292-8297, 1998; Honda et al., EMBO J. 18:22-27, 1999; Sharp et al., J. Bio. Chem. 274:38189-38196, 1999).

Mutations of conserved cysteine residues or deletions in the Mdm2 RING finger domain abolish its ability to target p53 ubiquitination and prolong Mdm2's half-life (Fang et al., J Biol Chem 275:8945-51, 2000; Honda and Yasuda, Oncogene 19:1473-6, 2000). Since Mdm2 is capable of self-ubiquitination (Honda et al., EMBO J. 18:22-27, 1999) and is degraded by the proteasome pathway with kinetics that are similar to those of p53, it is unclear how the intrinsic ubiquitin ligase activity of Mdm2 may be preserved during p53 ubiquitination. Such a mechanism would be expected to distinguish between Mdm2 self-ubiquitination and the ubiquitination of its NH2-terminal associated protein, p53.

The side chains of lysil residues serve as a conjugation site for ubiquitin, as well as for the small ubiquitin-like modifier protein SUMO-1 (Johnson et al., EMBO J. 16:5509-5519, 1997; Kamitani et al., J. Biol. Chem. 272:28557-28562, 1997). Covalent attachment of SUMO-1 (also termed Sentrin, Ubl1, or, in yeast, Smt3) to lysines was demonstrated for the RanGAP1 (Ran GTPase activating protein1) (Mahajan et al., Cell 88:97-107, 1997), IkBα (Desterro et al., Mol.Cell 2:233-239,1998), PML (Kamitani et al., J. Biol. Chem. 273:26675-26682,1998) and p53 (Gostissa et al., EMBO J. 18:6462-6471,1999: Rodriguez et al., EMBO J. 18:6455-6461, 1999). SUMO-1, which is 18% identical to ubiquitin, utilizes a similar conjugation pathway consisting of activation by a heterodimeric Aos1-Uba2 activating enzyme (E1) and conjugation onto a substrate by Ubc9 (Johnson et al., J. Biol. Chem. 272:26799-26802, 1997; Johnson and Blobel, J. Biol. Chem. 272:26799-26802, 1997), a protein with a strong sequence similarity to ubiquitin-conjugating enzymes (E2s). Unlike ubiquitin, SUMO-1 does not conjugate to itself and thus is not capable of forming chains similar to the poly-ubiquitin, which are required for recognition by the 26S proteasome.

Although SUMO-1 is an essential gene in Saccharomyces cerevisiae, the function of SUMO-1 modification (sumoylation) is not well understood. SUMO-1 conjugation is implicated in altering the subcellular localization of its RanGAP1, Sp100 and PML substrates (Mahajan et al., Cell 88:97-107, 1997; Duprez et al., J. Cell Sci. 112:381-393, 1999; Stemdorf et al., J. Biol. Chem. 274:12555-12566, 1999), in regulating septin ring dynamics during the cell cycle in budding yeast (Johnson and Blobel, J. Cell Biol. 147:981-993, 1999), and in neural differentiation in Drosophila (Lehembre et al., Mol. Cell. Biol.20:1072-1082,2000). Sumoylation of p53 is believed to increase its transcriptional activities (Gostissa, et al., EMBO J. 18:6462-6471, 1999; Rodriguez et al., EMBO J. 18:6455-6461, 1999). SUMO-1 has an entirely different effect on IkBa, where it becomes attached to the major ubiquitination site (Lysine 21; Desterro et al., Mol. Cell 2:233-239, 1998), thereby preventing ubiquitination and protecting sumoylated IkBa from proteasome-dependent proteolysis.

RING finger domains in E3 ubiquitin ligases were demonstrated to recruit E2 ubiquitin conjugating enzymes (Lorick et al., Proc. Natl. Acad. Sci. USA 96:11364-11369, 1999; Chen et al., J Biol Chem. In Press, 2000). The facts that Ubc9 is highly homologous to ubiquitin conjugating enzymes, that Mdm2 binding partner p53 is sumoylated, and that one of the critical sumoylation sites of PML is located within its RING finger domain prompted us to examine the possibility that Mdm2 serves as a substrate for SUMO-1 conjugation.

### SUMMARY OF THE INVENTION

The present invention provides a method for modulating Mdm2 activity by altering the level of sumoylation of Mdm2 protein, e.g., at amino acid 446 of Mdm2. In a specific embodiment Mdm2 activity increases the sumoylation of Mdm2 protein, increases the stability of Mdm2, and increases ligase activity of Mdm2 E3. In a further embodiment, decreasing the sumoylation of Mdm2 produces a decrease in the stability of Mdm2. In a further embodiment, a method of modulating Mdm2 activity where the sumoylation of Mdm2 is decreased comprises increasing the ubiquitination of Mdm2.

As sumoylation of Mdm2 affects its stability and ligase activity, the invention also provides a method of regulating p53 activity. This method comprises modulating sumoylation of Mdm2, where the sumoylation of Mdm2 regulates ubiquitination of p53 and alteration of the ubiquitination of p53 results in regulation of p53 activity. In particular, decreasing sumoylation of Mdm2, thereby decreasing ubiquitination of p53, results in increased p53 activity. In a specific embodiment, the decrease in sumoylation of Mdm2 comprises increasing the ubiquitination of Mdm2. Preferably, increased ubiquitination is produced by p19^{ARF}. In a further embodiment, increased phosphorylation of Mdm2 is produced, preferably by p38. In an additional embodiment, decreased sumoylation is produced by inhibiting binding of Ubc9 to the Ubc9 binding domain on Mdm2. Preferably, the Ubc9 binding domain on Mdm2 comprises amino acids 40-59 on Mdm2.

The invention provides a method of detecting sumoylation of Mdm2. This method comprises detecting the presence of sumoylated Mdm2 isolated from a mixture comprising mixing Mdm2 protein, SUMO-1, Aos1/Uba2, and Ubc9, where the mixture is incubated for a time sufficient for sumoylation of Mdm2 to occur under control conditions. In a specific embodiment, the SUMO-1 is radiolabeled. In a further embodiment, the radiolabel is ³⁵S. Alternatively, in a specific embodiment, the SUMO-1 is epitope tagged. In a further embodiment, the SUMO-1 is a hemagluttinin (HA)-tagged SUMO-1. In another embodiment, the mixture is a cell-free mixture.

The method of detection of the sumoylation of Mdm2 may further comprise culturing cells that have been transfected with an expression vector that encodes Mdm2 and an expression vector that encodes SUMO-1, under conditions that permit expression of sufficient quantities of Mdm2 and SUMO-1 to allow for detection of sumoylated Mdm2. In a further embodiment, the cells may be lysed and the lysates may be clarified prior to detection for the presence of sumoylated Mdm2.

An assay system for identifying a test compound that regulates sumoylation of Mdm2 e.g., in vitro, also is provided. This system comprises Mdm2 protein, SUMO-1, Aos1/Uba2, and Ubc9, wherein the level of Mdm2 protein is sufficient to detect Mdm2 sumoylation. This assay system can be used for identifying a test compound that regulates sumoylation of Mdm2. In one embodiment, the components are cell free. In an alternative embodiment, the assay system comprises cells that have been transfected with an expression vector that encodes Mdm2 and an expression vector that encodes SUMO-1, where the population of transfected cells expresses sufficient quantities of Mdm2 and SUMO-1 to allow for detection of sumoylated Mdm2.

The present invention also provides for a method for identifying a test compound that modulates sumoylation of Mdm2, which method comprises detecting a difference in the level of sumoylation of Mdm2 in an assay system contacted with a test compound, where a difference in the level of sumoylation of Mdm2 indicates that the test compound selectively modulates sumoylation of Mdm2. In one embodiment, the method uses an assay system is cell free. In an alternative embodiment, the method comprises detecting a difference in the level of sumoylation of Mdm2 in an assay system comprising cells transfected with an expression vector encoding Mdm2 and an expression vector encoding SUMO-1, where the population of transfected cells expresses sufficient levels of Mdm2 and SUMO-1 to detect Mdm2 sumoylation, where one population of transfected cells is contacted with a test compound, and where a difference in the level of sumoylation of Mdm2 in the population of cells contacted with the test compound compared to a population of cells not contacted with the test compound indicates that the test compound modulates sumoylation of Mdm2.

A method for detecting the amount of sumoylated Mdm2 in a sample, where the method comprises probing for sumoylated Mdm2 in an immunoprecipitate of Mdm2 from a sample also is provided by the present invention.

The present invention further provides for a method of treating a condition of uncontrolled cell growth, where the method comprises inhibiting sumoylation of Mdm2. In a specific embodiment, the condition of uncontrolled cell growth is a cancer. In one embodiment, the cancer is associated with a decrease in p53 activity in the cancer cells. In a further embodiment of a method of treating cancer comprises treating the cancer with a p53 gene therapy. In another embodiment, the inhibition of sumoylation of Mdm2 as a method of treating a condition of uncontrolled cell growth comprises increasing ubiquitination of Mdm2.

### DESCRIPTION OF THE DRAWINGS

FIGS. 1A and 1B. SUMO-1 is conjugated to Mdm2 in vivo. A. Whole cell extracts (WCE) prepared from normal human fibroblasts were immunoprecipitated (IP; 500 μg) with monoclonal antibodies either to Mdm2 or to SUMO-1 and analyzed by immunoblotting with the indicated antibodies. B. 293T cells were transfected with Flag-Mdm2 and HA-SUMO-1. WCE prepared 24 h later were immunoprecipitated (IP) using monoclonal antibodies against Mdm2 and analyzed by immunoblotting using monoclonal antibodies to HA or to Flag as indicated in Figure. Right part of the Figure depicts the expression level of the transfected constructs.

FIGS. 2A and 2B. Mutation of Lys446 in Mdm2 abolishes SUMO-1 conjugation. A. 293T cells were transfected with the indicated Flag-tagged Mdm2 constructs and HA-SUMO-1. WCE were IP with antibodies indicated in Figure. The two bottom panels verify expression of the respective exogenously expressed constructs. The top panel shows conjugation of wildtype (wt) Mdm2 as well as K1 (K446R), K2 (K453R) and K3 (K465,466,468,469,472R) mutants to SUMO-1. B. Purified GST-tagged Mdm2-forms (wt or K1/K2/K3 mutants as in legend to 2A) were sumoylated in vitro using in vitro translated ³⁵S-labeled SUMO-1. The expected MW of the sumoylated Mdm2 is 120 kDa, taking into consideration the added MW of the GST moiety. After the sumoylation reaction GST-bound proteins were separated on SDS-PAGE and visualized by Coomassie staining (bottom panel). Upper panel shows an autoradiograph of sumoylated Mdm2 forms. Efficient sumoylation of Mdm2 was seen with the wt, K2 or K3 mutants but not with the K1 mutant form or in the absence of SUMO-1. Reduced sumoylation was seen in the absence of Ubc9 because of the presence of minute amounts of the Ubc9 in the in wheat germ extract used to perform in vitro translation.

FIGS. 3A-3C. Sumoylation at Lys446 of Mdm2 abolishes its self-ubiquitination and stability. A. Wt or K1 mutant forms of Mdm2 were subjected to the sumoylation reaction using purified SUMO-1, Aos1/Uba2 and Ubc9 as detailed in Experimental Procedures. SUMO-1- (or mock-) modified forms of Mdm2 were than subjected to the self-ubiquitination assay using ³²P-ubiquitin in the presence of purified E1 and E2. Following the ubiquitination reaction glutathione bead-coupled proteins were washed and proteins were separated on 8% SDS-PAGE. The lower panel (B) depicts Mdm2-protein visualized by Coomassie staining. The upper panel (A) shows an autoradiograph of ³²P-ubiquitin conjugated to the respective Mdm2 forms. B. 293T cells were transfected with the wt or K1 mutant form of Mdm2 and HA-Ub. WCE was subjected to immunoprecipitation using monoclonal antibodies to the Flag-tag. The lower panel shows expression level of the transfected Mdm2 forms. The upper panel depicts poly-HA-Ubiquitin chains conjugated to the respective Mdm2 forms. C. p53/Mdm2 double null cells were transfected with the wt or K1 forms of Mdm2 and 24 h later were subjected to pulse chase labeling with ³⁵S-methionine in the absence or presence of lactacystin (25 μM) as indicated in Figure. Proteins prepared at the indicated time-points were immunoprecipitated using monoclonal antibodies to Mdm2 followed by autoradiography. The upper part of the figure depicts analysis of the wt Mdm2 transfected cells whereas the lower part shows analysis of the K1 expressing cells.

FIGS. 4A-4C. Mdm2 sumoylation does not affect its association with p53. A. The association of Mdm2 with p53 is not affected by its sumoylation. Immunoprecipitation of p53 (pAb421) or of Mdm2 (2A10 to C- and N20 to N-terminal domains) followed by immunoblot analysis using either of the Mdm2 antibodies (upper panel) or p53 antibodies (lower panel) identified the association of p53 with Mdm2 in its SUMO-1-modified form and in its non sumoylated form. B. Mdm2 in its wt or K1 forms associates with p53 in vitro. In vitro translated, ³⁵S-labeled, Flag-tagged Mdm2 wt or K1 forms were incubated with HA-tagged p53 followed by immunoprecipitation with antibodies to either p53 (HA) or Mdm2 (Flag). Immunopurified material was separated on SDS-PAGE and subjected to autoradiography. Immunoprecipitations identified both the target and associated protein, respectively, as indicated by arrows on right panel. C. Wt or K1 forms of Mdm2 associates with p53 in vivo. Forced expression of Flag-tagged Mdm2 wt or K1 and HA-tagged p53 in Mdm2/p53 double null cells was followed by immunoprecipitation and immunoblot analysis with the antibodies indicated in the Figure.

FIGS. 5A-5D. Mdm2 sumoylation or mutation on Lys446 increases p53 ubiquitination and degradation and decreases p53 ability to mediate apoptosis. A. Mdm2/p53 double null cells were transfected with p53 and increasing amounts of the wt or the K1 forms of Mdm2 as indicated. WCE was immunoprecipitated using monoclonal p53 antibodies. The lower panel (B) shows expression levels of wt or K1 forms of Mdm2 detected by monoclonal Mdm2 antibodies. The upper part of the Figure (A) shows the relative decrease in p53 expression levels upon Mdm2 (wt vs. K1) expression. B. Ubiquitinated Mdm2 is less efficient in targeting the ubiquitination of p53. Bacterially expressed Mdm2 (3 mg) was subjected to in vitro ubiquitination (self ubiquitination) in the presence of E1 [45 ng], E2 UbcH5b [1.5 μg] and ubiquitin [3 μg; SIGMA] and 2 μM ATP in ubiquitination buffer for the 0, 2, or 3 h (to ensure efficient self-ubiquitination) as indicated in Figure. To monitor the degree of Mdm2's ability to target the ubiquitination of p53, equal amounts of the self-ubiquitinated Mdm2 were extensively washed with TBS/0.5% Triton X-100 before incubated with bacculovirus-expressed and purified form of HA-tagged p53 for 2 h in the presence of E1, E2 and ubiquitin. The degree of p53 ubiquitination was monitored via immunoblot analysis using antibodies to HA. C. Wt or K1 mutant forms of GST-Mdm2 (1 mg) were sumoylated (in the presence of purified E1 (Aos1/Uba2; 15 ng), E2 (Ubc9; 0.5 μg) and SUMO-1 (1 μg) or mock treated before incubation for 1 h with WCE (1 mg) prepared from 293T cells that over express exogenous form of HA-p53. GST-Mdm2-p53-bound beads were washed with TBS/0.5% Triton X-100 to minimize non-specific binding before initiating the in vitro ubiquitination reaction in the presence of E1 [15 ng], E2 UbcH5b [0.5 mg] and ubiquitin [1 mg; SIGMA] and 2 mM ATP in ubiquitination buffer. Proteins were separated on 10% SDS-PAGE. The ubiquitinated HA-p53 was detected by monoclonal HA-antibodies. Similar observations were made using HA-tagged p53 that was purified from bacculovirus-expressing Sf9 cells (data not shown). D. DNA fragmentation analysis of Mdm2/p53 double null cells co-transfected with wild-type p53, increasing concentrations of wt or K1 forms of Mdm2, and GFP plasmids. Percent of apoptotic GFP-positive cells is indicated in the Figure.

FIGS. 6A and 6B. Mdm2 sumoylation is reduced after cell exposure to radiation. A. Proteins prepared from control (-UV) or 2 h after UV-irradiation were incubated with bacterially expressed Mdm2 and in vitro translated ³⁵S-labeled SUMO-1 under conditions required to enable Mdm2 sumoylation (for 1 h or 2 h as indicated in Figure). The degree of SUMO-1 conjugation was monitored via autoradiography of the SDS-PAGE (upper panel), whereas the amount of Mdm2 in its respective forms was visualized via Coomassie blue staining (lower panel). Compare changes between control and UV treatment at the same time point, respectively. B. Normal human fibroblasts were treated with sham, UV, or X-rays at indicated doses in the presence of proteasome inhibitor (MG132, 40 mM, for 3 hr before protein preparation). Proteins, prepared at the indicated time points after exposure, were immunoprecipitated with antibodies to Mdm2 (IF2; which were used in this experiment, rather than 2A10 antibodies used in former experiments, since the latter do not recognize Mdm2 after exposure to radiation) and subjected to immunoblot analysis using antibodies to SUMO-1 (upper part of the figure). Middle part of the figure depicts analysis of the same membrane following its incubation with antibodies to Mdm2. Lower part of the figure shows immunoblot of p53 in extracts (100 μg) prepared from cells subjected to the same treatment yet in the absence of the proteasome inhibitor.

FIG. 7. Model for the regulation of Mdm2 ubiquitin-ligase activity by SUMO-1. Mdm2 is subjected to either ubiquitination or sumoylation. When self-ubiquitinated, the ubiquitin ligase activity of Mdm2 for p53 is impaired. Upon SUMO-1 conjugation, Mdm2 is protected from ubiquitination and elicits increased ubiquitin ligase activity, as reflected in increased ubiquitination and degradation of p53.

FIGS. 8A and 8B. A. Purified GST-Ubc-9 was incubated with the indicated in vitro translated ³⁵S-labeled Mdm2 forms for 1 h on ice. Glutathione-beads were added for 30 min. Beads were extensively washed (3× with TBS/0.5% Triton-X-100) and the proteins remaining on the washed beads were eluted in 3× Laemmli buffer. Proteins were separated by 10% SDS-PAGE and analyzed by autoradiography. The upper part shows the input of in vitro-translated Mdm2 forms in a pull-down assay. The lower part shows the association of the Mdm2 forms with Ubc-9 after GST-pull-down. B. Mdm2/p53 double null cells were transfected with the indicated Mdm2 plasmids and HA-tagged Sumo-1. Cells were harvested 36 h later and 500 μg of protein were immunoprecipitated using Mdm2 antibodies (2A10). The immunoprecipitates were washed and the proteins loaded onto 10% SDS-PAGE. Immunoprecipitated Mdm2 was detected using monoclonal Mdm2 antibodies (2A10, left). The same membrane was reprobed using monoclonal antibodies against Sumo-1 (Zymed, right). Bottom panel shows binding of Ubc9 to wt Mdm2 but not Mdm2 that is missing aa 40-59.

FIGS. 9A and 9B. A. A peptide corresponding to aa 40-59 on Mdm2 binds Ubc9. mu9bs (Mdm2 Ubc9 binding site) corresponding to aa 40-59 of Mdm2 and its control scrambled counterpart peptide mu9sb (were immobilized on an AminoLink column [Pierce]). 50 μl of peptide-bound beads were incubated with 1 mg of cell lysates from EJ mouse fibroblast cells. Beads were washed extensively with 0.5M LiCl/TBS and eluates were separated on a 14% gel. B. Ubc9 binds to mu9bs in vivo. NIH 3T3 cells were transfected with wt or mutant Ubc9 binding site peptide. 36 h after transfection cells were lysed and lysates were immunoprecipitated with HA-antibodies directed against the HA-tag of transfected peptides. Upper panel depicts to Co-immunoprecipitation of Ubc9 with the wt but not the mutant peptide. Lower panel shows expression level of transfected peptides.

FIGS. 10A-10D. A. Time dependent Mdm2 sumoylation in vitro. Bacterially expressed and purified GST-Mdm2 was subjected to in vitro sumoylation reaction in the presence of E1, Ubc9, and ³⁵S-Sumos. Reaction was terminated after 10,30,50 or 60 minutes, Proteins were separated on 8% gel, Commassie stained, dryed and exposed overnight (upper panel). Lower panel depicts Commassie blue staining of Mdm2 forms. B. A peptide corresponding to the Ubc-9 binding site on Mdm2 decreases Mdm2 in vitro sumoylation of Mdm2 in a time-dependent manner. (Upper part) The specific Ubc-9-binding site peptide or a nonspecific peptide was added to the reaction for the indicated time periods. The upper panel shows a radiograph of ³⁵S-labeled Sumo-1 conjugation to Mdm2 under the indicated condition. The lower panel depicts a Coomassie staining visualizing relative amounts of sumoylated and non-sumoylated forms of purified GST-Mdm2. C. The Mdm2 sumoylation assay was carried out in the presence of Ubc-9-binding site peptide (mu9bs) or a nonspecific peptide (mu9bs) which were added to the reaction at the indicated concentrations. The upper panel shows a radiograph of ³⁵S-labeled Sumo-1 conjugation to Mdm2 under the indicated condition. The lower panel shows a Coomassie staining visualizing relative amounts of sumoylated and non-sumoylated forms of purified GST-Mdm2. D. A peptide corresponding to the Ubc-9 binding site on Mdm2 decreases Mdm2 sumoylation in vivo. Mdm2/p53 double null cells were transfected with Flag-tagged Mdm2, HA-tagged Sumo-1 and increasing amounts of the specific or nonspecific Ubc-9-peptides, respectively. Cells were harvested 36 h later and 500 μg of protein was immunoprecipitated using Mdm2 antibodies (2A10). Immunoprecipitated Mdm2 levels were detected using 2A10 antibodies (top). The same membrane was stripped and reprobed using monoclonal antibodies against Sumo-1 (Zymed, middle) to visualize the level of Sumo-1 conjugation to Mdm2 after Ubc-9-peptide expression. The bottom panel shows the expression level of the respective Ubc-9 peptides.

FIG. 11. Ubc9 binding affects the form of Mdm2 associated with p53. Mdm2/p53 double null cells were transfected with mu9sb or the wild type form mu9bs together with p53 and Mdm2. 36 h after transfection protein extracts were prepared and used for immunoprecipitation of p53 (using D01 antibodies). Immunoprecipitated material was analyzed via immunoblotting using antibodies to Mdm2, enabling to reveal the two major forms of Mdm2, indicated. Western blotting with antibodies to HA revealed expression of the peptides whereas immunoblot with p53 depicts changes in levels of p53 expression. Wt Mdm2 and K1 (mutant at aa 446) forms were used as controls.

FIGS. 12A and 12B. A. Mutant Ubc9 decreases Sumoylation and increases ubiquitination of Mdm2 in vitro Bacterial expressed and purified GST-Mdm2 was incubated with purified wt or mutant Ubc9, 32P-labeled-Sumo1 and E1. Sumoylation assay was carried out as described in Material & Methods (upper panel). Half of the sumoylation reaction was subjected to additional ubiquitination reaction in the presence of HA-Ub, UbcH5b and E1 (detailed in Material & Methods), middle panel). Lower panel shows the sumoylated and non-sumoylated forms of Mdm2 in straight western depicted by Mdm2 antibodies. B. Mutant Ubc9 decreases Mdm2 Sumoylation, increases Mdm2 ubiquitination and decreases p53 ubiquitination in vivo. Normal human fibroblasts (TIG) were transfected with HA-Ub and wt or mutant Ubc9 plasmids as indicated. Cells were harvested and lysed 36 h after transfection. 750 mg cell lysates were immunoprecipitated with either Mdm2 or p53 antibodies. Left upper panel depicts degree of Mdm2 sumoylation after transfection of different Ubc9 constructs. Lower panel shows Mdm2 ubiquitination in same samples detected by HA antibodies. Right upper panel reflects dose dependent effect of Ubc9 mutant expression on p53 ubiquitination. Lower right panel reflects p53 levels upon western blot analysis.

FIG. 13A-13E. A. Bacterially expressed and purified Mdm2 (1 μg) was incubated with ³²P-labeled Sumo-1, purified Aos1/Uba2 and Ubc9 (0.5 μg) for 60 min at 37° C. in conjugation buffer. For indicated time-periods in vitro translated wt or mutant p19^{ARF} (0.5 mg) was added to the sumoylation reactions. Upper panel depicts the amount of Sumoylated Mdm2, lower panel reveal the level of Mdm2 and Sumo-Mdm2 in Coomassie blue gel. B. Bacterially expressed and purified Mdm2 (1 μg) was incubated with in vitro-translated ³⁵S-Sumo, purified Aos1/Uba2 and Ubc9 (0.5 μg) for 60 min at 37° C. in conjugation buffer in the presence of the increasing amounts of bacculovirus-expressed p19^{ARF} (0, 0.1, 0.5, 1 μg; lanes 1-4 respectively). Bead-bound complexes were washed before being denatured in 3× Sample Buffer. Proteins were separated on 8% SDS-PAGE. Gel was stained with Coomassie blue to verify equal Mdm2 input (lower panel), dried and exposed to X-ray film, which depicts SUMO-1 conjugation to Mdm2 in the presence of the indicated p19 forms (upper panel). Efficiency of in vitro Sumoylation is estimated to be around 20-30% based on the amount of Sumoylated Mdm2 relative to non-sumoylated form seen in Coomassie blue gels (see panels A&B). Based on amount of p19^{ARF} added a molar ratio of 1:1 is estimated to be required for inhibition of Mdm2 sumoylation by p19^{ARF}. C. p19^{ARF} decreases Mdm2 sumoylation in Mdm2/p53-/-cells. Double-null cells were transfected with Flag-tagged Mdm2 (1.5 μg), HA-tagged SUMO (1.5 μg) and p19 wt (1.5 μg). Cells were treated with MG132 (40 mM) for 5 h prior to protein preparation. For immunoprecipitations, proteins (500 μg) were incubated with Mdm2 antibodies (2A10) at 4° C. overnight before addition of protein A/G beads. Immunoprecipitated material was washed and proteins were loaded onto 10% SD S-PAGE, followed by immunoblot analysis with the indicated antibodies. (Left) Immunoprecipitated Mdm2 was detected using monoclonal Mdm2 antibodies (2A10, left). The same membrane was reprobed using SUMO antibodies (Zymed, right). (Bottom) Control for p19 expression was performed using polyclonal p19^{ARF} antibodies. D. Expression and binding of different p119^{ARF} forms to Mdm2. 293T cells were transfected with Flag-tagged Mdm2 (2 μg) and wt of D62 forms of p19^{ARF} constructs (2 μg). Cells were lysed and immunoprecipitation (about 1 mg of cell lysate) was carried out using Mdm2 antibodies (2A10). (Left) Expression control of transfected p19^{ARF} constructs. (Right) Expression of Mdm2 in immunoprecipitated material (top). Co-immunoprecipitation of p19^{ARF} with Mdm2 (bottom). E. p19^{ARF} expression attenuates Mdm2 sumoylation in p19-/-EJ cells. EJ cells were transfected with Flag-tagged Mdm2 (2 μg) and p19^{ARF} constructs (2 μg). Before harvesting, cells were treated for 5 h with MG132 (40 μM). Cells were lysed as described and immunoprecipitation (500 μg of protein extract) was performed using Mdm2 antibodies (2A10). SUMO-conjugation to Mdm2 was detected using monoclonal antibodies to SUMO-1 (top). The same membrane was reprobed using 2A10 antibodies directed against Mdm2 (middle). Immunoblot analysis was also performed to verify levels of p19^{ARF} expression (bottom).

FIG. 14A-14E. A. In vitro ubiquitination of Mdm2 is inhibited by p19^{ARF}. GST-Mdm2 was subjected to an in vitro ubiquitination reaction using purified forms of E1, E2 and Ub-HA in the presence of ATP. Addition of bacculovirus-purified p19^{ARF} at the indicated concentrations blocked Mdm2 ubiquitination. Glutathione bead-coupled GST-Mdm2 in its ubiquitinated or non-ubiquitinated forms was washed and analyzed on immunoblots using antibodies to HA (upper panel). Middle panel depicts the level of Mdm2 expression and the lower panel, the level of p19^{ARF} added to the reaction. B. p19^{ARF} expression increases Mdm2 self-ubiquitination in EJ cells. EJ cells were transfected with the indicated plasmids. Cells were harvested 36 h later and proteins (500 m μg) were immunoprecipitated using Mdm2 antibodies (2A10). Immunoprecipitated material was washed and proteins loaded onto 10% SDS-PAGE, followed by immunoblot analysis with HA-antibodies to detect covalent HA-ubiquitin conjugation to Mdm2 (upper panel). Middle panel depicts level of immunoprecipitated Mdm2. Lower panel represents western analysis of p19^{ARF} expression levels. C. In vitro ubiquitination of Mdm2 in the presence of p19^{ARF} and protein extracts. GST-Mdm2 was subjected to in vitro ubiquitination as indicated in (a) except that protein extracts prepared from EJ cells prior to and 2 h after UV irradiation (30 J/m2) were added to the reaction at the indicated concentrations. D. p19^{ARF} expression decreases p53 ubiquitination in EJ cells. EJ cells were transfected with Flag-tagged Mdm2 (1 μg), wt p53 (1 μg), HA-Ub (1 μg) and the relevant p19^{ARF} constructs (1 μg). Cells were harvested 36 h later and proteins (500 μg) were immunoprecipitated using monoclonal pAb421 antibodies. Immunoprecipitated material was washed and subjected to immunoblot analysis with HA-antibodies to detect covalent HA-ubiquitin conjugation to p53 (top). The level of p53 immunoprecipitated was monitored in immunoblots using polyclonal antibodies to p53 (middle). Control for p19^{ARF} expression using polyclonal antibodies against p19^{ARF} (bottom). E. p19^{ARF} expression increases p21-luc activity. EJ cells were transfected with the indicated plasmids. Luciferase activity shown was normalized with respect to β-galactosidase levels to adjust for transfection efficiency.

FIG. 15A-15D. A. Pharmacological inhibitor of p38 inhibitors attenuate p19^{ARF}-mediated Mdm2 ubiquitination in vivo. EJ cells were transfected with Flag-Mdm2 (2 μg), p19^{ARF}(2 μg) and HA-Ub (2 μg). 24 h after transfection cells were treated with indicated pharmacological inhibitors (PD 98059, 50 μM; SB203580, 10 and 100 μM, LY294002, 50 μM; Herbimycin, 1 μM). Mdm2 was immunoprecipitated and equal amounts were separated on a 8% SDS-PAGE. Upper panel depicts efficiency of Mdm2 ubiquitination by covalent binding of HA-Ubiquitin to Mdm2. Lower panel shows control for Mdm2 immunoprecipitation (2A10 antibodies). B. Phosphorylation of Mdm2 by p38 enables Mdm2 self-ubiquitination. Bacterially expressed Mdm2 or bacculovirus produced p19^{ARF} were subjected to p38 kinase reaction. Phosphorylated proteins were used for in vitro ubiquitination assay. (Top) HA-ubiquitin conjugation to Mdm2. Lower panels shows input levels of Mdm2 and p19^{ARF}. C. Protein extracts from ASKΔN expressing cells increase Mdm2 self-ubiquitination in vitro. Bacterially expressed and purified Mdm2 (1 μg) was incubated with cell lysates (25 μg) prepared from cells transfected with indicated plasmids (4 μg). In vitro ubiquitination assay was carried out as described above. Upper panel shows conjugation of HA-ubiquitin to Mdm2 (exposure time 20 sec). Lower panel depicts Mdm2 levels. D. Mdm2 mutated at Ser 218 is not capable of undergoing p19^{ARF}-mediated ubiquitination. Bacterially expressed and purified GST-Mdm2 wild type and S218A mutant forms (1 mg) were phosphorylated by p38 as indicated. Phosphorylated Mdm2 was subjected to in vitro ubiquitination reaction in the presence of p19^{ARF}. Analysis was carried out as detailed above.

FIG. 16A-16F. A. Activation of p38 increases Mdm2 ubiquitination in vivo. EJ cells were transfected with indicated plasmids. 36 h after transfection cells were harvested and Mdm2 was immunoprecipitated (2A10 antibodies). Upper panel shows ubiquitination of Mdm2 detected with the aid of HA antibodies directed against HA ubiquitin. Middle panels show expression levels of Mdm2 and p19^{ARF}. Lower panels depict phospho-and total p38 levels. B. Mutation at Ser218 of Mdm2 abolishes p38 induced Mdm2 ubiquitination. Normal human fibroblasts were transfected with indicated plasmids. 36 h after transfection cells were harvested and lysed. Mdm2 was immunoprecipitated and proteins were separated on a 8% SDS-PAGE. Upper panel depicts Mdm2 ubiquitination (exposure time 1 min), whereas lower panels shows levels of Mdm2, and p38 in its phosphorylated and total forms. C. Mutation at Ser218 of Mdm2 abrogates p19^{ARF}-induced Mdm2 degradation. NHF were transfected with wt or 218 mutant forms of Mdm2 and 24 h later pulse chase analysis was carried out as described in methods. Cells were lysed at indicated time points and Mdm2 forms were immunoprecipitated using Flag-antibodies. Immunoprecipitates were separated on a 8% SDS-PAGE. Gel was dried and exposed overnight to X-ray film. D. Activation of p38 decreases Mdm2 sumoylation in vivo. EJ cells were transfected with indicated plasmids and 36 h later cells were harvested. Lysates were immunoprecipitated with 2A10 antibodies directed against Mdm2. Immunoprecipitates were separated on 8% SDS-PAGE. Upper panels depict Mdm2-Sumo conjugates (detected via Sumo-1 anitbodies), as well as the 75 kD and 90 kD forms of Mdm2 (detected via Mdm2 antibodies). Lower panels depict levels of p19^{ARF}, phosphorylated FORM and total p38. E. Increased ubiquitination of Mdm2 after UV treatment is attenuated by SB203580. Cells were transfected with indicated plasmids. Where indicated, cells were treated with the pharmacological inhibitor of p38 (SB203580; 10 μM or with the inhibitor of P13K kinases (LY294002; 50 μM), 2 h prior to harvest. Cell pellets were lysed and extracts were subjected to immunoprecipitation using monoclonal antibodies against Mdm2 (2A10). Upper panel depicts characteristic poly-ubiquitination as shown by HA-antibodies. Lower panel shows levels of Mdm2 in the immunoprecipitated material. F. Increased p19^{ARF} binding to Mdm2 is after UV-treatment. Normalized amounts of Mdm2 (shown in second panel and based on Mdm2 expression levels shown in non-normalized top panel) were immunoprecipitated using monoclonal antibodies against Mdm2 (2A10). Western blot analysis was carried out using either Mdm2 (2A10) or p19^{ARF} (gift of C. Sherr) antibodies. To monitor possible changes in amount of p19 bound to Mdm2 association was monitored using normalized Mdm2 levels at the indicated time-points after UV-treatment. Middle panel shows immunoprecipitated Mdm2, bottom panel depicts p19^{ARF} associated with immunopreciptated Mdm2.

FIGS. 17A and 17B. A. p19^{ARF} decreases Ubc9 binding to Mdm2. Bacterially expressed and purified GST-Mdm2 (1 μg) was incubated with ³⁵S-labeled in vitro translated p19^{ARF} and Ubc9 at the indicated ratios. After 30 min of incubation GSH beads bound-GST-Mdm2 were washed and separated on a 12% SDS-PAGE. Gel was dried and subjected to autoradiograph, depicting relative amounts of Ubc9 and p19^{ARF} bound to Mdm2. B. Bacterially expressed and purified GST-Mdm2 (1 μg) was phosphorylated by purified form of active p38 kinase as indicated. Phosphorylated or un-phosphorylated Mdm2 was incubated for 30 min with in vitro translated UbcH5b and Ubc9 (25 μl). Beads were washed and separated on a 12% SDS-PAGE. Gel was dried and exposed to X-ray film. Position of Ubc9 and UbcH5b is shown.

### DETAILED DESCRIPTION

Mdm2 is a RING finger E3 ubiquitin ligase that ubiquitinates the p53 tumor suppressor protein. The present invention is based, in part, on the discovery that Mdm2 is conjugated with SUMO-1 (sumoylated) at Lys446, which is located within the RING finger domain and plays a critical role in Mdm2 self-ubiquitination. Mutating Lys446 results in protecting Mdm2 from ubiquitination and degradation. Expression of Mdm2K446R (Lys446Arg mutation, yielding a mutant with a non-acylatable cationic group) leads to increased degradation of p53 and concomitant inhibition of p53-mediated apoptosis. In vitro sumoylation of Mdm2 abrogates its self-ubiquitination and increases its ubiquitin ligase activity toward p53. Increased E3 ligase activity of sumoylated Mdm2 is attributed to its protection from self-ubiquitination. In response to radiation, there is a dose- and time-dependent decrease in the degree of Mdm2 SUMO-1 modification, which is inversely correlated with the level of p53 expression. These results suggest that the maintenance of the intrinsic activity of a RING finger E3 ubiquitin ligase is sumoylation-dependent and that reduced Mdm2 sumoylation in response to DNA damage contribute to p53 stability.

The present invention is also based, in part, on the discovery that Ubc9 binds to a region of Mdm2 from about amino acid residue 40 to about amino acid residue 59. Binding of Ubc9 to a target protein is a necessary precondition to sumoylation of the target protein. This results suggests that compounds may be developed to inhibit Mdm2 sumoylation by blocking binding of Ubc9 to Mdm2.

Additionally, the present invention is also based on the discovery that p19^{ARF} modulates Ubc9 binding to Mdm2. Additionally, p38 modulates the phosphorylation state of Mdm2. Combined, studies indicate that p19^{ARF} and p38 interfere with Ubc9 induced sumoylation of Mdm2. Therefore, modulation of these systems may be a target for drug development.

As used herein, the term “activity” refers to the performance by a biomolecule of its function. For example, an activity of Mdm2 is ubiquitination of p53. An activity of p53 is induction of apoptosis and cell growth arrest. Activity also refers to the rate or ability to function.

“Mdm2” refers to the RING finger E3 ubiquitin ligase (NCBI Entrez Accession No. CAA78055; SWISS-PROT Accession Q00987; Oliner et al., Nature 1992,358:80) that ubiquitinates p53, among other functions. The amino acid sequence of Mdm2 is depicted in SEQ ID NO: 1.

“p53” refers to the tumor suppressor protein as this term is understood in the art.

“p19^{ARF}” refers to a Mdm2-regulating protein and is well understood in the art.

“SUMO-1” refers to the ubiquitin-like protein discussed in the Background and is used herein as this term is understood in the art. “Sumoylation” refers to ligation of SUMO-1 to a target protein. “Ubiquitination” refers to ligation of ubiquitin to a protein. In a specific aspect, sumoylation or ubiquitination (but not both) occur at lysine-446 (Lys446) of Mdm2.

“Ubc9” refers to a Sumo conjugating enzyme, which does not conjugate ubiquitin, and is discussed in the Background. As used here, this term is understood in the art. In one embodiment, a human Ubc9 is composed of 158 amino acids.

As used herein, the term “modulating” (and alternative grammatical forms, such as “modulate” and “modulation”) refers to altering, e.g., enhancing (increasing) or inhibiting (decreasing) the activity of a target, such as Mdm2. The present invention provides for modulating Mdm2 activity by targeting sumoylation of the protein. Sumoylated Mdm2 (or Mdm2 lacking Lys446) more effectively ubiquitinates p53, thus destabilizing that tumor suppressor protein. Conversely, leaving Lys446 free permits ubiquitination of Mdm2, destabilizing it and, consequently, enhancing p53 activity. Thus, modulation of Mdm2 sumoylation directly affects p53 activity. “Regulating p53 activity” refers to modulating sumoylation of Mdm2 to affect p53. In a preferred embodiment, e.g., for developing and implementing treatments for cancer, regulation of p53 activity means enhancing (or increasing) p53 activity.

Modulating Mdm2 activity can be achieved by a number of strategies, set forth below, including (i) identifying small molecule compounds, e.g., using the screening methods of the invention, that inhibit Mdm2 sumoylation; (ii) using peptides, such as a peptide corresponding to the sumoylation site of Mdm2, to inhibit sumoylation and/or increase Mdm2 ubiquitination; (iii) using peptides to increase ubiquitination and/or phosphorylation of Mdm2; (iv) targeting sumoylated Mdm2 for removal, e.g., by an intracellular antibody; (v) targeting SUMO-1 and other components of the sumoylation pathway, e.g., by antisense or intracellular antibodies; and (vi) combinations of the foregoing.

The screening and testing aspects of the invention have, at their basis, methods for detecting or determining sumoylation of Mdm2, since, as pointed out above, sumoylation of Mdm2 affects p53 activity. Such methods are also relevant for clinical testing. The term “method of detecting” sumoylation of Mdm2 refers to any biochemical or immunological technique, such as but by no means limited to differential molecular weight measurements, co-immunoprecipitation assays, sandwich immunoassays, and the like, to detect sumoylation of Mdm2. Screening and testing assays for identifying and developing drug candidates can be conducted in cell-free systems, cell-based systems, and transgenic animal systems. All of these systems are characterized by the presence of Mdm2, SUMO-1, and other proteins, such as Aos1/Uba2, and Ubc9, required for sumoylation to occur. In addition, the assays, particularly cell-free assays, are run in appropriately buffered ionic solutions, such that the reaction can occur.

The term “in vitro” refers to biological processes, usually, but not limited to, experimental conditions, that are performed outside the whole organism. The term in vitro can refer to cell-free and cell-based assay systems. The term “cell-free” refers to any system in which normal cellular reactions are reconstituted in the absence of cells. The term “cell-based” refers to any system where cellular reactions are performed in the presence of cellular components.

The term “control conditions” refers to conditions under which a given reaction, such as sumoylation or ubiquitination, can occur. Such conditions include permissive pH, ionic strength, adequate concentrations of reactants (target protein, i.e., Mdm2, and SUMO-1 or ubiquitin) and co-factors, and the like. The term control conditions reflects that these are positive control conditions for the reaction; in the presence of a test compound, such as a peptide or small molecule drug candidate (“test conditions”), the reaction may not occur.

As used herein, the term “tumor” refers to any unregulated cell growth. Tumors can be benign or malignant; malignant tumors are cancers. According to the present invention, various cancers can be treated, such as solid and soft tissue cancers. Soft tissue cancers include, but are not limited to, carcinomas and adenocarcinomas, e.g., head-and-neck, colorectal, ovarian, prostate, vulval, lung, and breast. Additional tumors include, but are not limited to, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, pancreatic cancer, squamous cell carcinoma, basal cell carcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, and retinoblastoma. The present invention is particularly useful in conjunction with p53-based therapies (see, e.g., U.S. Pat. No. 6,069,134 and PCT Publication Nos. WO 97/10007 and WO 95/12660), or to enhance the activity of endogenously expressed p53 in controlling cell growth.

A “test compound” is any molecule, including but not limited to peptide, polypeptide, protein, oligonucleotide, nucleic acid, natural product, synthetic product, carbohydrate, lipid, etc. that can be tested for its ability to modulate Mdm2 sumoylation (or ubiquitination) as set forth herein. Test compounds are discussed in more detail infra.

The invention will be better understood by reference to the following sections, directed to recombinant technology, screening assays, diagnostic methods, and therapeutic methods, as well as the accompanying Examples. This organization of the description into the various sections is intended to clarify the invention, and not limit it.

### Recombinant Technology

The present invention provides for expression of functional or mutant Mdm2 protein, SUMO-1, and, if required, sumoylation accessory proteins Aos1, p19^{ARF}, and Ubc9, for evaluation, diagnosis, or therapy.

In accordance with the present invention there may be employed conventional molecular biology, microbiology, and recombinant DNA techniques within the skill of the art. Such techniques are explained fully in the literature. See, e.g., Sambrook, Fritsch & Maniatis, Molecular Cloning: A Laboratory Manual, Second Edition (1989) Cold Spring Harbor Laboratory Press, Cold Spring Harbor, N.Y. (herein “Sambrook et al., 1989”); DNA Cloning: A Practical Approach, Volumes I and II (D. N. Glover ed. 1985); Oligonucleotide Synthesis (M. J. Gait ed. 1984); Nucleic Acid Hybridization [B. D. Hames & S. J. Higgins eds. (1985)]; Transcription And Translation [B. D. Hames & S. J. Higgins, eds. (1984)]; Animal Cell Culture [R. I. Freshney, ed. (1986)]; Immobilized Cells And Enzymes [IRL Press, (1986)]; B. Perbal, A Practical Guide To Molecular Cloning (1984); F. M. Ausubel et al. (eds.), Current Protocols in Molecular Biology, John Wiley & Sons, Inc. (1994).

A gene encoding Mdm2, any of the proteins of interest, such as Mdm2, whether genomic DNA or cDNA, can be isolated from any source, particularly from a human cDNA or genomic library. Methods for obtaining genes (i.e., coding sequences) are well known in the art, as described above (see, e.g., Sambrook et al., 1989, supra). The DNA may be obtained by standard procedures known in the art from cloned DNA (e.g., a DNA “library”), and preferably is obtained from a cDNA library prepared from tissues with high level expression of the protein, by chemical synthesis, by cDNA cloning, or by the cloning of genomic DNA, or fragments thereof, purified from the desired cell (See, for example, Sambrook et al, 1989, supra; Glover, D. M. (ed.), 1985, DNA Cloning: A Practical Approach, MRL Press, Ltd., Oxford, U.K. Vol. 1, II). Clones derived from genomic DNA may contain regulatory and intron DNA regions in addition to coding regions; clones derived from cDNA will not contain intron sequences. Whatever the source, the gene should be molecularly cloned into a suitable vector for propagation of the gene. Identification of the specific DNA fragment containing the desired Mdm2 gene may be accomplished in a number of ways. For example, a portion of an Mdm2 gene exemplified infra can be purified and labeled to prepare a labeled probe, and the generated DNA may be screened by nucleic acid hybridization to the labeled probe (Benton and Davis, Science 196:180, 1977; Grunstein and Hogness, Proc. Natl. Acad. Sci. U.S.A. 72:3961, 1975).

Additionally, the protein encoding nucleic acid sequence can be mutated in vitro or in vivo, to create and/or destroy translation, initiation, and/or termination sequences, or to create variations in coding regions and/or form new restriction endonuclease sites or destroy preexisting ones, to facilitate further in vitro modification. Such modifications can be made to introduce restriction sites and facilitate cloning the gene into an expression vector. Any technique for mutagenesis known in the art can be used, including but not limited to, in vitro site-directed mutagenesis (Hutchinson, C., et al., J. Biol. Chem. 253:6551, 1978; Zoller and Smith, DNA 3:479-488, 1984; Oliphant et al., Gene 44:177, 1986; Hutchinson et al., Proc. Natl. Acad. Sci. U.S.A. 83:710, 1986), use of TAB^{..} linkers (Pharmacia), etc. PCR techniques are preferred for site directed mutagenesis (see Higuchi, 1989, “Using PCR to Engineer DNA”, in PCR Technology: Principles and Applications for DNA Amplification, H. Erlich, ed., Stockton Press, Chapter 6, pp. 61-70).

The identified and isolated gene can then be inserted into an appropriate cloning vector. A large number of vector-host systems known in the art may be used. A wide variety of host/expression vector combinations may be employed in expressing the DNA sequences of this invention. Useful expression vectors, for example, may consist of segments of chromosomal, non-chromosomal and synthetic DNA sequences. Suitable vectors include derivatives of SV40 and known bacterial plasmids, e.g., E. coli plasmids col E1, pCR1, pBR322, pMal-C2, pET, pGEX (Smith et al., Gene 67:31-40, 1988), pMB9 and their derivatives, plasmids such as RP4; phage DNAS, e.g., the numerous derivatives of phage 1, e.g., NM989, and other phage DNA, e.g., M13 and filamentous single stranded phage DNA; yeast plasmids such as the 2μ plasmid or derivatives thereof; vectors useful in eukaryotic cells, such as vectors useful in insect or mammalian cells; vectors derived from combinations of plasmids and phage DNAs, such as plasmids that have been modified to employ phage DNA or other expression control sequences; and the like.

Recombinant molecules can be introduced into host cells via transformation, transfection, infection, electroporation, etc., so that many copies of the gene sequence are generated. Preferably, the cloned gene is contained on a shuttle vector plasmid, which provides for expansion in a cloning cell, e.g., E. coli, and facile purification for subsequent insertion into an appropriate expression cell line, if such is desired. For example, a shuttle vector, which is a vector that can replicate in more than one type of organism, can be prepared for replication in both E. coli and Saccharomyces cerevisiae by linking sequences from an E. coli plasmid with sequences form the yeast 2 μg plasmid.

A preferred expression host is a eukaryotic cell (e.g., yeast, insect, or mammalian cell). More preferred is a mammalian cell, e.g., human, rat, monkey, dog, or hamster cell. In specific embodiments, infra, the components of the assay system are expressed in a 293T cell. Other suitable cells include, without limitation, CHO, MDCK, COS, HeLa, 3T3, and other well known cells and cell lines. Alternatively, it is possible to transfect primary cells, including primary stem cells.

Yeast expression systems can also be used according to the invention to express any protein of interest. For example, the non-fusion pYES2 vector (XbaI, SphI, ShoI, NotI, GstXI, EcoRI, BstXI, BamH1, SacI, KpnI, and HindIII cloning sit; Invitrogen) or the fusion pYESHisA, B, C (XbaI, SphI, ShoI, NotI, BstXI, EcoRI, BamH1, SacI, KpnI, and HindIII cloning site, N-terminal peptide purified with ProBond resin and cleaved with enterokinase; Invitrogen), to mention just two, can be employed according to the invention.

Expression of the protein or polypeptide may be controlled by any promoter/enhancer element known in the art, but these regulatory elements must be functional in the host selected for expression. Promoters which may be used to control gene expression include, but are not limited to, cytomegalovirus (CMV) promoter (U.S. Pat. Nos. 5,385,839 and 5,168,062), the SV40 early promoter region (Benoist and Chambon, 1981, Nature 290:304-310), the promoter contained in the 3′ long terminal repeat of Rous sarcoma virus (Yamamoto, et al., Cell 22:787-797,1980), the herpes thymidine kinase promoter (Wagner et al., Proc. Natl. Acad. Sci. U.S.A. 78:1441-1445, 1981), the regulatory sequences of the metallothionein gene (Brinster et al., Nature 296:39-42, 20 1982); prokaryotic expression vectors such as the b-lactamase promoter (Villa-Komaroff, et al., Proc. Natl. Acad. Sci. U.S.A. 75:3727-3731, 1978), or the tac promoter (DeBoer, et al., Proc. Natl. Acad. Sci. U.S.A. 80:21-25, 1983); see also “Useful proteins from recombinant bacteria” in Scientific American, 242:74-94, 1980; promoter elements from yeast or other fungi such as the Gal 4 promoter, the ADC (alcohol dehydrogenase) promoter, PGK (phosphoglycerol kinase) promoter, alkaline phosphatase promoter; and transcriptional control regions that exhibit hematopoietic tissue specificity, in particular: beta-globin gene control region which is active in myeloid cells (Mogram et al., Nature 315:338-340, 1985; Kollias et al., Cell 46:89-94, 1986), hematopoietic stem cell differentiation factor promoters, erythropoietin receptor promoter (Maouche et al., Blood, 15:2557, 1991), etc.

For some purposes, it is preferable to produce the polypeptide in a recombinant system in which the protein contains an additional sequence tag that facilitates purification or identification, such as, but not limited to, a polyhistidine sequence or hemagluttinin, or a sequence that specifically binds to an antibody, such as FLAG, GST, a myc-tag, or hemagluttinin. The polypeptide can then be purified from a crude lysate of the host cell by chromatography on an appropriate solid-phase matrix or detected readily by ligand-specific or antibody specific binding.

### Screening and Chemistry

According to the present invention, nucleotide sequences derived from the gene encoding Mdm2, and peptide sequences derived from Mdm2, are useful targets to identify drugs that are effective in treating disorders associated with p53-regulated processes that are linked to increased sumoylation of Mdm2. Specifically, drugs that decrease the level of sumoylation of Mdm2, increase the level of ubiquitination and/or phosphorylation of Mdm2, and increase the activity of p53 are contemplated. Drug targets include without limitation (i) isolated nucleic acids derived from the gene encoding Mdm2; (ii) isolated peptides and polypeptides derived from Mdm2 polypeptides; and, most importantly, (iii) different receptors that regulate the sumoylation, ubiquitination, and phosphorylation of Mdm2.

In particular, the invention provides development of screening assays, particularly for high throughput screening of molecules that regulate the sumoylation, and thus the activity of Mdm2. Accordingly, the present invention contemplates methods for identifying test compounds that regulate the in vivo sumoylation of Mdm2 using various screening assays known in the art.

Any screening technique known in the art can be used to screen for agonists or antagonists of Mdm2 sumoylation, ubiquitination, and phosphorylation. The present invention contemplates screens for small molecule ligands or ligand analogs and mimics, as well as screens for natural ligands that agonize or antagonize Mdm2 sumoylation, ubiquitination, and phosphorylation in vivo. For example, natural products libraries can be screened using assays of the invention.

Another approach uses recombinant bacteriophage to produce large libraries. Using the “phage method” (Scott and Smith, Science 249:386-390, 1990; Cwirla, et al., Proc. Natl. Acad. Sci., 87:6378-6382, 1990; Devlin et al., Science, 49:404-406, 1990), very large libraries can be constructed (10⁶-10⁸ chemical entities). A second approach uses primarily chemical methods, of which the Geysen method (Geysen et al., Molecular Immunology 23:709-715, 1986; Geysen et al. J. Immunologic Method 102:259-274, 1987; and the method of Fodor et al. (Science 251:767-773, 1991) are examples. Furka et al. (14th International Congress of Biochemistry, Volume #5, Abstract FR:013, 1988; Furka, Int. J. Peptide Protein Res. 37:487-493, 1991), Houghton (U.S. Pat. No. 4,631,211, issued December 1986) and Rutter et al. (U.S. Pat. No. 5,010,175, issued Apr. 23, 1991) describe methods to produce a mixture of peptides that can be tested as agonists or antagonists.

In another aspect, synthetic libraries (Needels et al., Proc. Natl. Acad. Sci. USA 90:10700-4, 1993; Ohlmeyer et al., Proc. Natl. Acad. Sci. USA 90:10922-10926, 1993; Lam et al., International Patent Publication No. WO 92/00252; Kocis et al., International Patent Publication No. WO 9428028) and the like can be used to screen for Mdm2 ligands according to the present invention. Test compounds are screened from large libraries of synthetic or natural compounds. Numerous means are currently used for random and directed synthesis of saccharide, peptide, and nucleic acid based compounds. Synthetic compound libraries are commercially available from Maybridge Chemical Co. (Trevillet, Cornwall, UK), Comgenex (Princeton, N.J.), Brandon Associates (Merrimack, N.H.), and Microsource (New Milford, Conn.). A rare chemical library is available from Aldrich (Milwaukee, Wis.). Alternatively, libraries of natural compounds in the form of bacterial, fungal, plant and animal extracts are available from e.g. Pan Laboratories (Bothell, Wash.) or MycoSearch (NC), or are readily producible. Additionally, natural and synthetically produced libraries and compounds are readily modified through conventional chemical, physical, and biochemical means (Blondelle et al., Tib Tech, 14:60, 1996).

Knowledge of the primary sequence of Mdm2, combined with identification of the sumoylation (and ubiquitination) site at Lys 446 and Ubc9 binding site at a region from about amino acid residues 40 to about residue 59 can provide an initial clue as the inhibitors or antagonists of the protein. Identification and screening of agonists and antagonists is further facilitated by determining structural features of the protein in these regions, e.g., using X-ray crystallography, neutron diffraction, nuclear magnetic resonance spectrometry, and other techniques for structure determination. These techniques provide for the rational design or identification of agonists and antagonists of ubiquitination or sumoylation, respectively.

In a specific embodiment of the invention, an in vitro assay system is comprised of the Mdm2 protein, SUMO-1, and sumoylation cofactors. Specifically, the sumoylation cofactors are Aos1/Uba2 and Ubc9. In another specific embodiment, and in vivo assay system comprised of cells, that are transfected with expression vectors that encode Mdm2 and SUMO-1, are cultured under conditions that allow for expression of both proteins. Both systems allow for the identification of compounds that regulate sumoylation of Mdm2.

In a specific embodiment, the present invention provides for inhibiting sumoylation of Mdm2 by targeting binding of Ubc9 to Mdm2. Amino acid residues 40-59 of Mdm2 are necessary for Ubc9 binding; Ubc9 binding to a target is necessary for sumoylation to occur. Thus, inhibiting binding of Ubc9 to Mdm2 residues 40-59 will result in decreased sumoylation of Mdm2. In one embodiment, inhibition of Ubc9 binding is produced by addition of p19^{ARF} and/or increased phosphorylation of Mdm2. The phosphorylation of Mdm2 may be produced by any protein kinase, preferably p38. In one embodiment, p38 phosphorylates Mdm2 at serine 218.

The present invention contemplates numerous approaches to block Ubc9 binding to Mdm2. One strategy employs peptides corresponding to all or a part of the sequence of Mdm2 from about amino acid 40 to about amino acid 59. A second strategy employs peptidomimetics corresponding to such peptides. Such peptidomimetics can be prepared using traditional synthetic techniques, by rational drug design, or a combination thereof. Similarly, small molecules can be developed, e.g., using the screening assays disclosed herein, that block binding of Ubc9 to Mdm2. Still another method employs an intracellular antibody the blocks binding of Ubc9 to Mdm2. Alternatively, addition of p19^{ARF} and/or phosphorylation of the Mdm2 protein also may decrease sumoylation of Mdm2. Other strategies known in the art can also be employed.

### Methods of Diagnosis

According to the present invention, genetic variants of Mdm2 can be detected to diagnose a Mdm2 associated disease state, such as increased susceptibility soft tissue cancer formation. The various methods for detecting such variants are described supra. Where such variants impact Mdm2 function, either as a result of a mutated amino acid sequence, which may lead to increased protein sumoylation, they are expected to result in decreased p53 activity. The present invention also contemplates detecting the level of Mdm2 protein that is sumoylated in tissues compared to other tissues. A high level of sumoylated Mdm2 protein may lead to an expected decrease in p53 activity. The level of sumoylated Mdm2 protein may be detected using numerous strategies in the art such as, but not limited to, chromatography, spectrometry, gel electrophoresis, immunoassay, Lowry assay, and detection of protein labels.

### Nucleic Acid Assays

The DNA may be obtained from any cell source. Non-limiting examples of cell sources available in clinical practice include without limitation blood cells, buccal cells, cervicovaginal cells, epithelial cells from urine, fetal cells, or any cells present in tissue obtained by biopsy. Cells may also be obtained from body fluids, including without limitation blood, plasma, serum, lymph, milk, cerebrospinal fluid, saliva, sweat, urine, feces, and tissue exudates (e.g., pus) at a site of infection or inflammation. DNA is extracted from the cell source or body fluid using any of the numerous methods that are standard in the art. It will be understood that the particular method used to extract DNA will depend on the nature of the source. Generally, the minimum amount of DNA to be extracted for use in the present invention is about 25 pg (corresponding to about 5 cell equivalents of a genome size of 4×10⁹ base pairs). Sequencing methods are described in detail, supra.

In another alternate embodiment, RNA is isolated from biopsy tissue using standard methods well known to those of ordinary skill in the art such as guanidium thiocyanate-phenol-chloroform extraction (Chomocyznski et al., Anal. Biochem., 162:156, 1987). The isolated RNA is then subjected to coupled reverse transcription and amplification by polymerase chain reaction (RT-PCR), using specific oligonucleotide primers that are specific for a selected site. Conditions for primer annealing are chosen to ensure specific reverse transcription and amplification; thus, the appearance of an amplification product is diagnostic of the presence of a particular genetic variation. In another embodiment, RNA is reverse-transcribed and amplified, after which the amplified sequences are identified by, e.g., direct sequencing. In still another embodiment, cDNA obtained from the RNA can be cloned and sequenced to identify a mutation.

### Protein Assays

In an alternate embodiment, biopsy tissue is obtained from a subject. Antibodies that are capable of distinguishing between different polymorphic forms of Mdm2 are then contacted with samples of the tissue to determine the presence or absence of a Mdm2 polypeptide specified by the antibody. The antibodies may be polyclonal or monoclonal, preferably monoclonal. Measurement of specific antibody binding to cells may be accomplished by any known method, e.g., quantitative flow cytometry, or enzyme-linked or fluorescence-linked immunoassay.

### Therapeutic Uses

According to the present invention, administration of compounds that decrease Mdm2 sumoylation, e.g., compounds that increase Mdm2 ubiquitination at Lys446 or decrease Mdm2 sumoylation at Lys446, may be used as a treatment option in patients tumors or cancers that are resistant to current and conventional treatments. Mdm2 activity can be regulated by methods, such as, but not limited to, (i) administration of compounds or peptides that block sumoylation of Mdm2 protein, (ii) increasing the ubiquitination of Mdm2 protein and (ii) treatment with p53 gene therapy. Preferably, methods for decreasing the sumoylation of Mdm2, described in this application, are exclusive of radiation treatments.

Using the screening methods described above, compounds that are determined to decrease p53 ubiquitination may be used for treatment purposes. The test compounds, salts thereof, antibodies, and antisense constructs may be formulated into pharmaceutical compositions. The pharmaceutical composition comprises a therapeutically, inhibiting preventing, or blocking effective amount of at least one of the above. The pharmaceutical compositions also typically include a pharmaceutically acceptable carrier (or dosing vehicle), such as ethanol, glycerol, water, and the like. Examples of such carriers and methods of formulation are described in Remington's Pharmaceutical Sciences, 18th edition (1990), Mack Publishing Company. The pharmaceutical composition may also include other additives, such as a flavorant, a sweetener, a preservative, a dye, a binder, a suspending agent, a colorant, a disintegrant, an excipient, a diluent, a lubricant, a plasticizer, or any combination of any of the foregoing.

Treatment of tumors with p53 gene therapy involves transferring a vector comprising a gene for a functional p53 into target cells of a subject suffering from a tumor that is characterized by decreased p53 activity. It is also preferred that the p53 coding sequence is operatively associated with a promoter that permits high level expression in human cells, and preferably in tumor cells.

To be effective, enough p53 vector must be delivered so that enough cells must be transformed with an p53 gene therapy vector to overcome the decreased activity of endogenous p53. The determination of the dose of an p53 gene therapy vector depends on the type of vector, how it is delivered, and the susceptibility and receptivity of the subject. All of these factors can be determined by routine dosing methods well known in the art. In the practice of the present invention, anti-tumor gene therapy refers to administration or delivery of a gene encoding p53, either alone or in combination with other genes effective for treating tumors.

Examples of anti-tumor gene therapies of the prior art include, but are by no means limited to, introduction of a suicide gene; introduction of an apoptosis gene; introduction of a tumor suppresser gene; and introduction of an oncogene antagonist gene. Preferably anti-tumor genes, such as p53, are supplemented with immunostimulatory genes to enhance recruitment and activation of immune effector cells. If a viral, such as adenovirus, vector is used (see, e.g., PCT Publication No. WO 95/14101), the presence of adenoviral antigens could also provide an adjuvant effect to overall enhanced immune responsiveness. Gene therapy in accordance with the invention can be used to treat any cancer, but particularly tumors with decreased p53 activity. Preferably, p53 is increased in the tumor cells of the cancer.

As discussed above, a vector is any means for the transfer of a nucleic acid according to the invention into a host cell. Preferred vectors for transient expression are viral vectors, such as retroviruses, herpes viruses, adenoviruses and adeno-associated viruses. Thus, a gene encoding a functional p53 protein or polypeptide domain fragment thereof can be introduced in vivo, ex vivo, or in vitro using a viral vector or through direct introduction of DNA. Expression in targeted tissues can be effected by targeting the transgenic vector to specific cells, such as with a viral vector or a receptor ligand, or by using a tissue-specific promoter, or both. Targeted gene delivery is described in PCT Publication No. WO 95/28494.

Intracellular antibodies (sometime referred to as “intrabodies”) have been used to regulate the activity of intracellular proteins in a number of systems (see, Marasco, Gene Ther. 4:11, 1997; Chen et al., Hum. Gene Ther. 5:595, 1994), e.g., viral infections (Marasco et al., Hum. Gene Ther. 9:1627, 1998) and other infectious diseases (Rondon et al., Annu. Rev. Microbiol. 51:257, 1997), and oncogenes, such as p21 (Cardinale et al., FEBS Lett. 439:197-202, 1998; Cochet et al., Cancer Res. 58:1170-6, 1998), myb (Kasono et al., Biochem Biophys Res Commun. 251:124-30, 1998), erbB-2 (Graus-Porta et al., Mol Cell Biol. 15:1182-91, 1995), etc. This technology can be adapted to inhibit Mdm2 activity by expression of an anti-Mdm2 intracellular antibody.

In a specific embodiment, antisense nucleic acids, which may be used to inhibit expression or translation of Mdm2 or Ubc9, particularly to suppress uncontrolled cell growth, such as seen in cancers. An “antisense nucleic acid” is a single stranded nucleic acid molecule which, on hybridizing with complementary bases in an RNA or DNA molecule, inhibits the latter's role. For example, this technology can be adapted to inhibit translation of Ubc9 and thus block the role of Ubc9 in sumoylation of Mdm2.

In another embodiment, peptides that block the sumoylation of Mdm2 also may be used to suppress uncontrolled cell growth, as seen in cancers. The peptide may be of any sequence that modulates Mdm2 sumoylation. For example, a peptide fragment comprising the Ubc9 binding domain on Mdm2 (amino acids 40-59 of Mdm2) may be used. The peptide fragment may compete with the full length protein to interact with Ubc9. This competition may be used to decrease the sumoylation of Mdm2. These peptides may include sequences that increase delivery of the protein into the cell (as described below), decrease degradation of the protein, or the like.

### Translocation Peptide Sequences

Peptide sequences have been identified that mediate membrane transport, and accordingly provide for delivery of polypeptides to the cytoplasm. For example, such peptides can be derived from the antennapedia homeodomain helix 3 to generate membrane transport vectors, such as penetratin (PCT Publication WO 00/29427; see also Fischer et al., J. Pept. Res. 2000, 55:163-72; DeRossi et al., Trends in Cell Biol. 1998, 8:84-7; Brugidou et al., Biochem. Biophys. Res. Comm. 1995, 214:685-93). Protein transduction domains, which include the antennapedia domain and the HIV TAT domain (see Vives et al., J. Biol. Chem. 1997, 272:16010-17), posses a characteristic positive charge, which led to the development of cationic 12-mer peptides that can be used to transfer therapeutic proteins and DNA into cells (Mi et al., Mol. Therapy 2000, 2:339-47).

Therapeutic polypeptides can be generated by creating fusion proteins or polypeptide conjugates combining a translocation peptide sequence with a therapeutically functional sequence. For example, p21^{WAFI}-derived peptides linked to a translocation peptide inhibited ovarian tumor cell line growth (Bonfanti et al., Cancer Res. 1997, 57:1442-1446). These constructs yield more stable drug-like polypeptides able to penetrate cells and effect a therapeutic outcome. These constructs can also form the basis for rational drug design approaches.

### EXAMPLES

The present invention will be better understood by reference to the following Examples, which are provided as exemplary of the invention, and not by way of limitation.

### Example 1

### Sumoylation of Mdm2 Affects Its Stability and Ligase Activity

This Example demonstrates that Mdm2 is sumoylated and that Mdm2 stability and ligase activity are altered as a consequence of SUMO-1 conjugation (sumoylation).

### Materials and Methods

Expression vectors. Human wild type Mdm2 cDNA was amplified by PCR (HiFi Taq polymerase, GIBCO) and uni-directionally cloned, with the addition of two N-terminal Flag-tags, between the EcoRI and BamHI sites of pcDNA3. Bacterial expression vector of GST-Mdm2 (in pGEX-4T-1; Pharmacia) was kindly provided by Dr. A. Weissman. Wild type Mdm2 in pcDNA3 or pGEX-4T-1 was used as a backbone for generating the site-directed mutagenesis (Quick Change, Stratagene) to substitute lysines 446 (K1), 453 (K2) or 465, 466, 468, 469, 472 (K3) for arginines. The integrity of the resulting constructs was verified by sequencing. HA-tagged p53 expression vector was previously described (Fuchs et al., Oncogene 17:2543-2547, 1998). pcDNA3-HA-SUMO-1 expression vector and PCMV-HA-Ub were previously described (Rodriguez et al., EMBO J. 18:6455-6461, 1999, Treier et al., Cell 78:787-798, 1994).

Cells.

293T, adenovirus-transformed human embryo kidney cells, p53/Mdm2 double null cells, and normal human fibroblasts (TIG) were maintained in DMEM (GIBCO), supplemented with 10% heat-inactivated fetal bovine serum and antibiotics in 5% CO₂. Transfections were performed by the calcium phosphate precipitate technique for 293T cells and by lipofection (LipofectaminPlus, GIBCO) for p53/Mdm2 null cells. The medium was changed 6 h after transfection and cells were harvested 24 h later.

Antibodies.

Monoclonal antibodies against Flag- and HA-epitopes were purchased. (M2, SIGMA, and HA11, BabCo). Antibodies (monoclonal) against SUMO-1 were purchased (Zymed). Monoclonal antibodies 2A10, N20 (Santa Cruz) and IF2 (Oncogene) were used for Mdm2 analysis. p53 detection was carried out using monoclonal pAb421 antibodies (Oncogene) and polyclonal FL393 antibodies (SantaCruz).

In vitro Conjugation of SUMO-1.

Glutathione-beads (SIGMA) were used to purify bacterially expressed forms of GST-Mdm2 forms (wt, K1, K2, K3) as previously described (Lorick et al., Proc. Natl. Acad. Sci. USA 96:11364-11369, 1999). Bacterially expressed purified Mdm2 (1-2 μg) proteins were incubated with either purified form of bacterially expressed SUMO-1 or with in vitro translated ³⁵S-labeled SUMO-1 (TNT, Promega), as indicated in Results. To initiate SUMO-1 conjugation to Mdm2, purified Aos1/Uba2 (15 ng) and Ubc9 (0.5 μg) were added to the reaction mix for 30 min at 37° C. in conjugation buffer (20 mM Hepes, pH 7.4, 5 mM MgCl₂, 2 mM ATP, 10 mM creatine phosphate, 1 unit creatine phosphokinase). Bead-bound SUMO-1-modified Mdm2 was either taken for subsequent reaction (see p53 ubiquitination) or washed (3× TBS/0.5% Triton-X-100) before denaturation for 5 min at 95° C. in 3× Sample Buffer. Proteins were separated on 8% SDS-PAGE. The gel was stained with Coomassie blue, dried and exposed to X-ray film (Xomat, Kodak).

Mdm2 Self-ubiquitination Assay.

Purified GST-Mdm2 wt or mutant forms were sumoylated (or mock treated with the respective buffers as control) by addition of the purified form of bacterially expressed SUMO-1 together with Aos1/Uba2 and Ubc9 for 30 min at 37° C. in conjugation buffer (20 mM Hepes, pH 7.4, 5 mM MgCl2, 2 mM ATP, 10 mM creatine phosphate, 1 unit creatine phosphokinase). Upon its SUMO-1 modification, Mdm2-bound to glutathione beads was washed and incubated for 1 h at 37° C. in ubiquitination buffer (50 mM Tris-HCl, pH 8.0, 5 mM MgCl₂, 0.5 mM DTT, 2 mM NaF, 3 mM okadaic acid) supplemented with ³²P-labeled HA-tagged ubiquitin (Tan et al., Mol. Cell. 3, 527-533, 1999), purified E1 (15 ng), UbcH5b (0.5 μg) and ATP (2 mM). Glutathione-bound GST-Mdm2 beads were extensively washed (3× with TBS/0.5% Triton-X-100). To analyze Mdm2 self-ubiquitination, the proteins remaining on the washed beads were eluted in Laemmli buffer, separated on 8% SDS-PAGE and analyzed by autoradiography and Coomassie Blue staining.

In vitro Ubiquitination of p53.

Purified GST-Mdm2 forms that were sumoylated (using the purified form of SUMO-1, Aos1/Uba2 and Ubc9 as indicated above) or mock treated (with all reagents except SUMO-1), were incubated for 1 h on ice with HA-p53 that was either affinity purified from Sf9 cells infected by bacculovirus expressing HA-p53 or with protein extracts prepared from 293T cells that over-express HA-p53. Bead-bound SUMO-1 modified Mdm2-p53 complex was washed (3× with PBS/0.5% Triton-X-100) before incubation with purified E1 (15 ng), Ubc9 (0.5 μg), ATP (2 mM) and ubiquitin (1 mg, SIGMA) for 30 min at 37° C. Glutathione-bound GST-Mdm2-p53 beads were washed (3× with PBS/0.5% Triton-X-100), eluted by Laemmli sample buffer, separated by 10% SDS-PAGE and analyzed by immunoblotting using a monoclonal HA-antibody.

In vivo Half-life Measurements.

p53/Mdm2 double null cells were transfected with wt or K1 forms of Mdm2 expression vectors (4 μg). ³⁵S-methionine (1 mCi) was added to the cell cultures 24 h after transfection for 5 hr (pulse) followed by chase (2 mM cold methionine) for the indicated time-points. Protein extracts (1 mg) were used for immunopurification of Mdm2 using mixture of monoclonal antibodies (2A-10/3 F1). Immunoprecipitated Mdm2 was washed, separated on 10% SDS-PAGE and analyzed by autoradiography.

In vivo Sumoylation/Ubiquitination.

Cells were transfected with Mdm2 constructs and cDNAs encoding either HA-tagged SUMO-1 or HA-tagged ubiquitin to analyze sumoylation or ubiquitination, respectively. Harvested cell pellets were lysed by incubation with two volumes of 2% SDS in TBS (10 mM Tris-HCl (pH 8.0), 150 mM NaCl) at 95° C. for 10 min. Eight volumes of 1% Triton-X-100 in TBS were added, and lysates were sonicated for 2 min. The solution was incubated for 30 min at 4° C., with Protein A/G beads (GIBCO) and clarified by 30 min centrifugation (14,000 rpm) at 4° C. The protein concentration was determined by Bradford assay. For immunoprecipitation 500 mg of protein was incubated with respective antibodies at 4° C. overnight before Protein A/G beads (25 μl) were added for 2 h. The beads were washed with 0.5M LiCl in TBS followed by two additional washes with TBS. Proteins were loaded onto 10% SDS-PAGE, followed by immunoblot analysis with the indicated antibodies and ECL detection (Amersham).

Apoptosis Assay.

Mdm2/p53 double null cells were transfected with Mdm2 constructs (as indicated) and marker plasmid encoding Green Fluorescent Protein, pEGFP (1 μg). Twenty four hours after transfection cells were collected, fixed in 1% paraformaldehyde, treated with 70% ethanol, washed with PBS, treated with DNase free RNase A (1 mg/ml), and stained with propidium iodide (PI; 40 μg/ml). DNA fragmentation analysis of GFP-positive cells (50,000 per measurement in triplicates) was carried out on a flow cytometer (Becton Dickinson). The percentage of cells to the left of the diploid G0/1 peak, characteristic of hypodiploid cells, was calculated as the percentage of apoptotic cells.

### Results

Mdm2 is Conjugated with SUMO-1 in Human Cells.

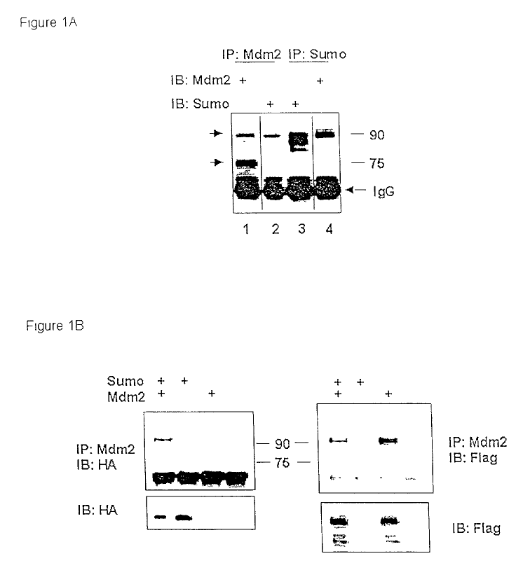

Analysis of Mdm2 expression by immunoblotting often reveals multiple forms that are detected by different Mdm2 antibodies. To test whether some of these forms could result from SUMO-1-modification of Mdm2, we monitored the possible presence of SUMO-1-conjugated Mdm2 in vivo. For this purpose, we immunoprecipitated Mdm2 or SUMO-1 from denatured normal human fibroblast cell lysates (to inhibit the activity of de-sumoylation enzymes) and analyzed the samples by immunoblotting with the monoclonal antibodies against Mdm2 and SUMO-1. Of the two major Mdm2-antibody-reacting bands (with apparent molecular weight of 75 kDa and 90 kDa; FIG. 1A, lane 1) only 90 kDa protein was recognized by SUMO-1 antibody (FIG. 1A, lane 4). SUMO-1 association with Mdm2 was confirmed by a reciprocal experiment in which proteins were first immunoprecipitated with Mdm2 antibody and analyzed in western blot with the SUMO-1 antibody (FIG. 1A, lane 2). These data demonstrate that endogenous Mdm2 with an apparent molecular weight of 90 kDa is conjugated with SUMO-1. The change in mobility of sumoylated Mdm2 revealed a ˜15 kDa shift, which suggests that Mdm2 modification can be attributed to the conjugation of a single SUMO-1 molecule.

To confirm this finding, we transfected 293T cells with Flag-tagged Mdm2 and HA-tagged SUMO-1 constructs. In these cells, we found that all detectable exogenous Mdm2 was of 90 kDa (FIG. 1B, right panel). Analysis of Mdm2 immunoprecipitates by HA antibody clearly showed that Mdm2 is conjugated with HA-SUMO-1 (FIG. 1B, left panel).

Lys446 is Required for Mdm2 Sumoylation.

The major lysine residue for SUMO-1 conjugation in PML protein is Lys65, the fifth residue distal to the beginning of the first loop of the RING finger (Kamitani et al., J. Biol. Chem. 273:26675-26682, 1998). Comparison of the RING finger motifs of PML and Mdm2 revealed the presence of a similarly distanced Lys446 within the RING finger of Mdm2. Analogously located lysines are found in other RING finger proteins, including murine Mdm2 (K444), equine Mdm2 (K446), BRCA1 (K32), Praja1 (K359) and AO7 (K144).

To determine which lysine residues within the RING finger is required for the sumoylation of Mdm2, we mutated the respective amino acids, generating three mutants; K1 (K446R), K2 (K453R) and K3 (K465,466,468,469,472R). Co-expression of HA-SUMO-1 and the lysine-mutated forms of Mdm2 revealed that K2 and K3 exhibit the same mobility as the wild type form of Mdm2, although expressed to somewhat lower levels. Conversely, the K1 mutant of Mdm2 migrated as a 75 kDa protein (FIG. 2A, middle panel). The latter resembles one of the endogenously expressed Mdm2 species (FIG. 1A). These data imply that the exogenously expressed wild type Mdm2 is efficiently sumoylated, whereas the K1 mutant is not. Furthermore, analysis of Flag immunoprecipitates by immunoblotting with HA antibody revealed that the K1 mutant is not conjugated with SUMO-1 in vivo (FIG. 2A, upper panel). Thus, Lys446 is the major sumoylation site on Mdm2. These findings also demonstrate that mutations on other lysines within the RING finger domain do not significantly impair Mdm2 sumoylation, although we cannot exclude the possibility that residual sumoylation.

We directly assessed the sumoylation of Mdm2 in vitro. Bacterially expressed wt or mutant forms of Mdm2 were incubated with purified Aos1/Uba2, Ubc9 and in vitro translated immunopurified ³⁵S-labeled SUMO-1 followed by SDS-PAGE and autoradiography (FIG. 2B, upper panel) or Coomassie Blue staining (FIG. 2B, lower panel). As evident from both the ³⁵S-detected signal and the mobility of Mdm2 proteins, SUMO-1 was conjugated with the wild type Mdm2, as well as with the K2 and the K3 forms, but not with the K1 mutant. These data provide direct evidence for Mdm2 sumoylation at Lys446, which is similar to one of the major PML sumoylation sites.

Sumoylation of Mdm2 Inhibits its Self-ubiquitination.

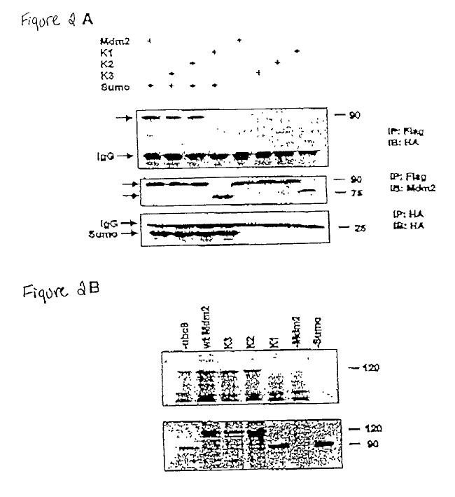

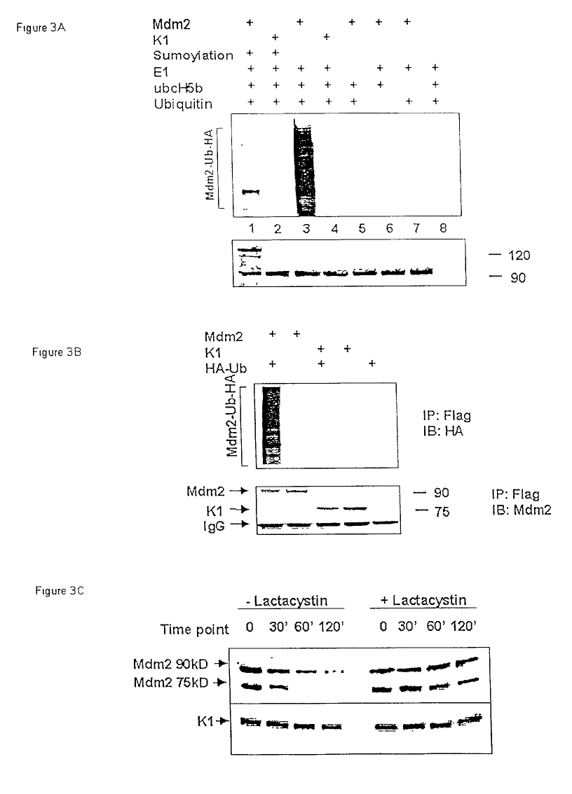

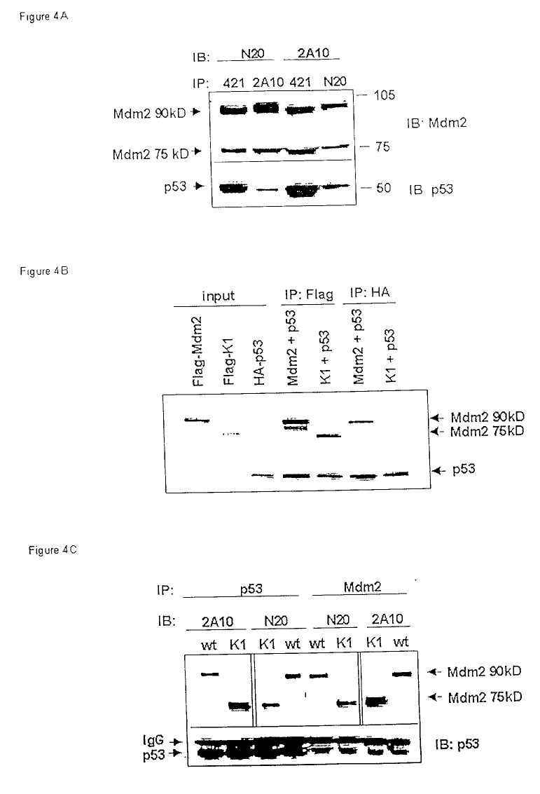

To investigate whether SUMO-1 and ubiquitin utilize a similar conjugation site, we carried out an in vitro ubiquitination reaction of the wild type or K1 mutant forms of Mdm2. Incubation of Mdm2 proteins with E1, E2 (UbcH5b), ³²P-ubiquitin and ATP resulted in detectable ubiquitination of wt Mdm2 but not of the K1 Mdm2 mutant (FIG. 3A, lane 3 vs. lane 4). This result demonstrates that Lys446 serves as a major site for Mdm2 self-ubiquitination. Our findings suggest that SUMO-1 may compete with ubiquitin for conjugation at Lys446.

We next examined whether SUMO-1-modification alters Mdm2 self-ubiquitination by performing the sumoylation reaction prior to in vitro ubiquitination. SUMO-1 modification of Mdm2 was performed with purified bacterially expressed components, including SUMO-1, Aos1/Uba2 and Ubc9. As shown in FIG. 3A, sumoylation of the wild type Mdm2 efficiently decreased the degree of Mdm2 self-ubiquitination (FIG. 3A, lane 1 vs. lane 3). The efficiency of Mdm2 sumoylation under these conditions was verified by slower migration of SUMO-1-Mdm2 (FIG. 3A, lower panel). Taken together, these data demonstrate that Lys446 serves as the primary ubiquitination site on Mdm2, and that Mdm2 sumoylation attenuates its self-ubiquitination in vitro. Lysine 446 is required for Mdm2 ubiquitination and degradation in vivo.

We next examined the role of Lys446 in Mdm2 ubiquitination in vivo. Expression of Flag-tagged Mdm2 and HA-tagged ubiquitin in 293T cells followed by Flag immunoprecipitation and anti-HA antibody analysis enabled the detection of ubiquitinated wild type Mdm2 (FIG. 3B). In contrast, K446R mutant was not ubiquitinated. These observations establish that Lys446 is required for Mdm2 ubiquitination in vivo.

To determine whether self-ubiquitination of Mdm2 at Lys446 is required for Mdm2 degradation in vivo, we measured Mdm2 half-life. ³⁵S-pulse-chase labeling experiments revealed that the 90 kDa form of Mdm2 is more stable compared with the 75 kDa form of Mdm2 (FIG. 3C). Whereas the half-life of the K1 form is around 2 h, the p90 sumoylated form of Mdm2 exhibits a half-life of less than 1 h as compared with a half-life of less than 30 min for the non-SUMO-1-modified p75 form of Mdm2. Treatment of the cells with proteasome inhibitor lactacystin prolonged the half-life of both p75 and p90, whereas the half life of the K1 remained unchanged (FIG. 3C). These observations further substantiate that conjugation of SUMO-1 to Mdm2 stabilizes Mdm2 by preventing its self-ubiquitination. It should be noted that since the Mdm2 of ˜90 kDa is sumoylated (FIGS. 1-2) and its direct ubiquitination may be impaired (FIG. 3A), it is likely that removal of SUMO-1 by sentrin-specific proteasel-like enzymes occurs prior to Mdm2 ubiquitination. In this case, the difference in the kinetics of p90 form of Mdm2 and K1 degradation is probably underestimated. Together with the data presented in FIG. 3B, these results suggest that Lys446 is required for Mdm2 ubiquitination and degradation in vivo.

Mdm2 Sumoylation does not Affect its Association with p53.

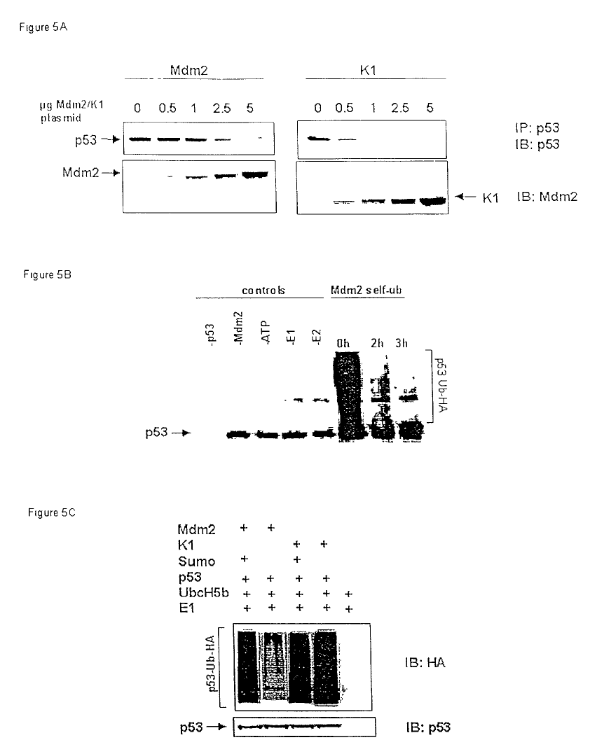

We next determined whether SUMO-1 conjugation to Mdm2 affects Mdm2 binding to p53. The association between p53 and Mdm2, in its sumoylated and non-sumoylated forms, was monitored in vivo and in vitro. The mutant and sumoylated forms of Mdm2 are equally efficient in its associating with p53 (FIG. 4A). The association between p53 and Mdm2 could be identified using antibodies to either the amino or the carboxyl terminus of Mdm2, thus excluding the presence of a spliced Mdm2 form (Perry et al., J. Biol. Chem. 275:5733-5738, 2000) in this complex. Each of Mdm2 forms tested was capable of associating with p53 in vitro as evident from co-immunoprecipitation of in vitro translated proteins (FIG. 4B). Forced expression of p53 and wt or the K1 forms of Mdm2 in p53/mdm2 double null cells also enabled the detection of efficient association between p53 and the wt (sumoylated in cells) or K1 (non-SUMO-1-modified) forms of Mdm2 (FIG. 4C). These results suggest that SUMO-1 modification does not affect Mdm2 association with p53.

Mdm2 K446R, Which is not Capable of Self-ubiquitination, is More Efficient than wt Mdm2 in p53 Degradation.

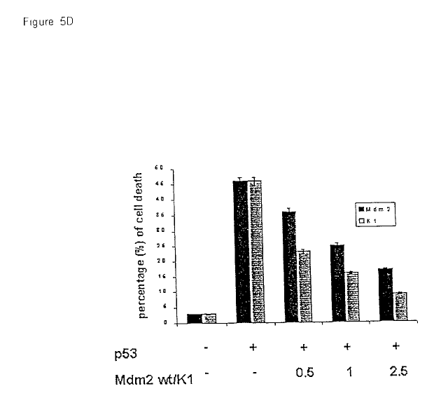

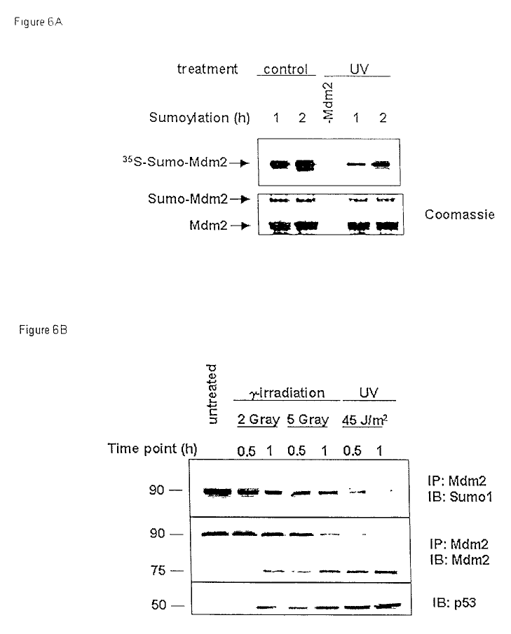

Mdm2 elicits the ubiquitin ligase activities towards itself as well as toward p53. Given the observations that Mdm2 sumoylation attenuates its self-ubiquitination, prolongs its half-life but does not affect its association with p53, we have examined the significance of Mdm2 self-ubiquitination in the Mdm2-mediated degradation of p53. To this end, we compared the effects of the wild type Mdm2 and the K1 mutant on p53 accumulation in a co-transfection assay (Haupt et al., Nature 387:296-299, 1997; Kubbutat et al., Nature 387:299-303, 1997). p53/Mdm2 double null cells (Jones et al., Nature 378:206-208, 1995) were used to exclude the influence of endogenous Mdm2. Forced expression of the wild type or the K1 forms of Mdm2 in p53/Mdm2 double null cells led to a dose-dependent decrease in the levels of p53 protein. Nevertheless, when compared with the wt form of Mdm2, five times lesser amounts of K1 expression construct were required to achieve the same decrease in the level of p53 (0.5 mg vs. 2.5 mg; FIG. 5A, upper panel). This result indicates that the Mdm2 mutant defective in self-ubiquitination is more efficient in promoting p53 degradation in vivo than the wild type Mdm2. Since, under the conditions of forced Mdm2 expression used in these experiments, the levels of K1 and wt forms of Mdm2 were similar (FIG. 5A, lower panel), we cannot explain the enhanced activity of K1 by merely the accumulation of a more stable K1 protein. Therefore, these data also suggest that Mdm2 self-ubiquitination is likely to be detrimental to Mdm2's ability to ubiquitinate p53.

Sumoylation Increases the Ability of Mdm2 to Ubiquitinate p53.

To directly assess the effect of Mdm2 ubiquitination on its ability to target the degradation of p53, we performed the Mdm2 self-ubiquitination reaction prior to adding Mdm2 to p53 ubiquitination assay. In vitro ubiquitination reactions were carried out using the purified E1, E2 (UbcH5b), Mdm2 (as the source for E3 ligase) and affinity purified HA-tagged p53 expressed in Sf9 insect cells. In this two-step reaction we enabled efficient self-ubiquitination of Mdm2 before addition of p53 as a substrate to assay Mdm2-targeted ubiquitination. Analysis of p53 ubiquitination (performed with antibodies to HA-tagged p53, which enabled distinguishing p53 from Mdm2 ubiquitination) revealed that in its ubiquitinated form, Mdm2 is less efficient in mediating the ubiquitination of p53 (FIG. 5B).

We have next assessed the effect of Mdm2 sumoylation on its ability to target the ubiquitination of p53. Whereas Mdm2 mediated noticeable p53 ubiquitination (which was dependent on the presence of E1, E2, ubiquitin and ATP; FIG. 5B and data not shown), there was a remarkable increase in the degree of p53 ubiquitination by SUMO-1-modified Mdm2 (FIG. 5C). An increased ubiquitin ligase activity of the K1 mutant correlates with its elevated ability to degrade p53 in vivo (FIG. 5A). Sumoylated Mdm2 was as efficient in p53 ubiquitination as the K1-mutant of Mdm2, which does not exhibit self-ubiquitination. These data suggest that whereas ubiquitination of Mdm2 impairs its ubiquitin ligase activity, Mdm2 sumoylation at Lys446 preserves Mdm2 ligase activity for p53 ubiquitination by blocking the self-ubiquitination of Mdm2 E3 ubiquitin ligase.

Mdm2 K446R, Which is not Capable of Self-ubiquitination, is More Efficient than wt Mdm2 in Inhibition of p53-induced Apoptosis.

To examine the biological consequences of accelerated p53 degradation by mutant Mdm2, deficient in self-ubiquitination, we compared the abilities of the K1 and wt forms of Mdm2 to inhibit p53-mediated apoptosis. Co-expression of wt or K1 forms of Mdm2 in Mdm2/p53 double null cells resulted in a dose-dependent decrease in the apoptosis induced by forced expression of p53. When compared with wt Mdm2, the K1 mutant, which is protected from self-ubiquitination, elicited a greater decrease in degree of cell death (FIG. 5D). This finding suggests that abolished self-ubiquitination renders Mdm2 more efficient in inhibiting p53-mediated programmed cell death.

Radiation Decreases the Degree of Mdm2 Sumoylation.

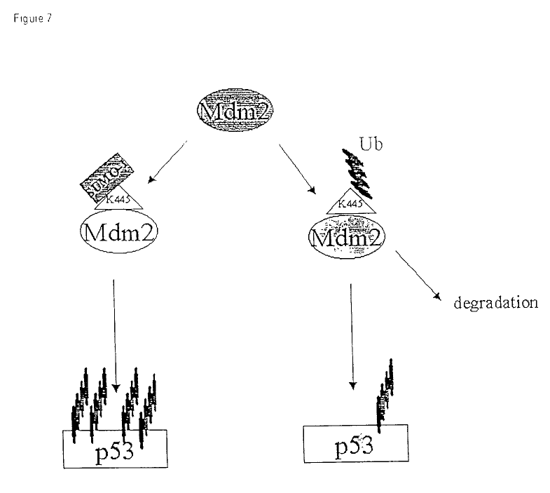

Stabilization of p53 in response to stress and DNA damage is imperative for its ability to coordinate the cellular stress response. Under these circumstances, increased p53 stability has been attributed, in part, to its dissociation from Mdm2 (Shieh et al., Cell 91:325-334, 1997, Fuchs et al., Oncogene 17:2543-2547, 1998). The current finding of Mdm2 sumoylation, and its effect on Mdm2 E3 ligase activity towards p53, point to an additional layer of regulation, which is independent of Mdm2 dissociation from p53. Given the effect of sumoylated Mdm2 on p53 ubiquitination, we determined whether DNA damage, which is among the best characterized stimuli that increase p53 stability, alters the relative amount of SUMO-1-modified Mdm2. Proteins prepared from UV-treated cells were tested for their ability to mediate Mdm2 sumoylation in vitro. When compared with proteins prepared from non-treated cells, the proteins derived from UV-treated cells were less efficient in eliciting Mdm2 sumoylation (FIG. 6A). These observations suggest that degree of Mdm2 sumoylation is reduced upon UV-irradiation. Mdm2 that has been subjected to sumoylation in the presence of proteins from UV-treated cells also elicited a less efficient ubiquitination of p53 than proteins prepared from sham-treated cells (data not shown).

To further assess the changes to Mdm2 sumoylation in response to radiation we have monitored the expression levels of the SUMO-1 modified Mdm2, the non-sumoylated form of Mdm2 and p53 in proteins prepared from UV- or X-ray treated cells. A clear time- and dose-dependent decrease in the amount of SUMO-1-modified Mdm2 after exposure to either UV or X-rays was seen both in immunoprecipitation of Mdm2 followed by SUMO-1 western (FIG. 6B, upper panel) and in straight immunoblot analysis of 90 kDa Mdm2 (FIG. 6B, middle panel). Immunoblot analysis of Mdm2 expression using proteins prepared after UV or X-ray treatment in the presence of proteasome inhibitors (to preserve the non-sumoylated form of Mdm2) revealed a shift from the sumoylated to the non-sumoylated forms of Mdm2, in a time- and dose-dependent fashion (FIG. 6B, middle panel). These results suggest that DNA damage, in the form of radiation, effectively diminishes the degree of Mdm2 sumoylation in a dose- and time-dependent manner. The decrease of Mdm2 sumoylation coincided with an increased level of p53 expression (FIG. 6B, lower panel), suggesting that the regulation of SUMO-1 modification of Mdm2 may play a role in p53 stabilization, in response to DNA damage. The temporal decrease in the amount of SUMO-1-conjugated Mdm2 point to one of the mechanisms by which p53 is no longer targeted by Mdm2 for ubiquitination in response to DNA damage.

### Discussion

A common characteristic of RING finger proteins that exhibit ubiquitin ligase activity is that they are capable of self-ubiquitination (Lorick et al., Proc. Natl. Acad. Sci. USA 96:11364-11369, 1999; Fang et al., J Biol Chem 275:8945-51, 2000; Honda and Yasuda, Oncogene 19:1473-6, 2000). While it is conceivable that self-ubiquitination is required to limit the duration and magnitude of ubiquitin ligase output, the mechanism distinguishing self-ubiquitination from the ubiquitination of the target substrate has been long sought. Such a mechanism would have been expected to provide a window of opportunity for substrate ubiquitination before self-destruction takes place. Here we demonstrate that self-ubiquitination of Mdm2 at Lys446 within its RING finger domain impairs Mdm2 ubiquitin ligase activity and ability to ubiquitinate p53. We also show that conjugation of the SUMO-1 ubiquitin-like protein to the same lysine residue abrogates Mdm2 self-ubiquitination and therefore preserves Mdm2 ubiquitin ligase activity, resulting in concomitantly increased efficiency of ubiquitination of its major substrate, p53 (FIG. 7). Our data provides additional evidence for the important role of ubiquitin-like proteins in the modulation of E3 ubiquitin ligase activities. Conjugation of Cullins with the ubiquitin-like protein Nedd8/RUB 1 is implicated in the biological activities of other RING finger-based E3 ubiquitin ligases such as SCF-ROC₁ and VCB-Cul2-ROC1 (Lammer et al., Genes Dev. 12:914-926, 1998; Kamura et al., Genes Dev. 13:2928-2933, 1999; Hori et al., Oncogene 18:6829-6834, 1998; Liakopoulos et al., Proc. Natl. Acad. Sci. USA 96:5510-5515, 1999). It is possible that like Mdm2 sumoylation, Nedd8/RUB1 conjugation is also capable of preserving ubiquitin ligase activity, although the biochemical mechanism of this modification has yet to be identified.

Sumoylation of PML or Sp100 proteins was reported to result in nuclear localization within PML bodies (Duprez et al., J. Cell Sci. 112:381-393, 1999; Sternsdorf et al, J. Biol. Chem. 274:12555-12566, 1999). Subcellular localization of Mdm2 is unlikely to be affected by its sumoylation since the localization of the K1 mutant that cannot undergo SUMO-1-modification did not differ from the localization of the wild type protein (data not shown). This finding suggests that protein sumoylation may result in multiple biological outcomes, varying with the nature of the sumoylated protein.

In the case of Mdm2, SUMO-1-modification competes with Mdm2 ubiquitination for the major conjugation site, Lys446. It was previously shown that similar competition between sumoylation and ubiquitination occurs at the Lys21 of IkBα (Desterro et al., Mol. Cell 2:233-239, 1998). Although resembling the case of IkBα in the role of the active interplay between sumoylation and ubiquitination dictating the degree of Mdm2 stability, SUMO-1 modification of Mdm2 has major consequences for its activity as ubiquitin ligase. Moreover, unlike the sumoylation of IkBα which was shown to occur in a small percentage of IkBa molecules (Desterro et al, Mol. Cell 2:233-239, 1998), SUMO-1-modification of Mdm2 was found in almost 100% of exogenously expressed proteins, and is largely noted among the endogenous Mdm2 species (as represented by the 90 kDa MW band). Indeed, Mdm2 has often been characterized as a ˜90 kDa protein (Chen et al., 1993; Mol. Cell. Biol. 16:2445-2452, 1996). Our experiments suggest that the non-sumoylated form of Mdm2, which was bound to p53 and was recognized by amino-terminal Mdm2 antibodies, is not a spliced form of Mdm2 which lacks N-terminal sequences (Perry et al., J Biol Chem 275:5733-5738, 2000). Given that sumoylation protects the Mdm2 protein from degradation, SUMO-1-modified Mdm2 is expected to be the more abundant Mdm2 form. That a large portion of Mdm2 molecules is sumoylated further emphasizes the biological significance of SUMO-1-modification for the Mdm2 protein.

Our findings demonstrate that sumoylation resulted in the inhibition of Mdm2 self-ubiquitination, thus providing the mechanism which enabled us to distinguish between substrate ubiquitination and self-ubiquitination. Accordingly, duration of protein sumoylation is expected to reflect the time frame during which the ubiquitination of Mdm2 targets (such as p53) occurs. A temporal decrease in Mdm2-sumoylation occurs in response to DNA damage and is inversely correlated with the elevated levels of p53. Attenuated Mdm2 sumoylation is likely to explain why Mdm2 is no longer capable of mediating the efficient ubiquitination of p53 in response to DNA damage, in addition to decreased p53-Mdm2 binding (Shieh et al., Cell 91:325-334, 1997, Fuchs et al., Oncogene 17:2543-2547, 1998).

Maintenance of a sumoylated substrate is expected to be regulated by the balance between Mdm2 sumoylation and removal of SUMO-1 by the de-sumoylating enzymes, including the sentrin-specific protease 1 (SENP1), which is distantly related to the yeast Smt3-specific protease ULP1 (Li and Hochstrasser, Nature 398:246-251, 1999). Regulation of Mdm2-sumoylation may also be affected by Mdm2 phosphorylation as well as by some of its associated proteins, including p14^{ARF}, p300, pRb, and E2F1.

The consequences of Mdm2 sumoylation, as shown in this study, are reflected in increased ubiquitination and degradation of p53 and the diminished p53 ability to elicit programmed cell death, which results in attenuated activities of this tumor suppressor protein.

Accordingly, sumoylation is likely to play a key role in Mdm2's ability to elicit its oncogenic activities.

Sumoylation of the Mdm2 target substrate p53 was shown not to affect p53 stability and was found on only a small portion of p53 molecules (Gotissa et al., EMBO J.

18:6462-6471, 1999; Rodriguez et al., EMBO J. 18:6455-6461, 1999), suggesting that SUMO-1-modification of Mdm2 may have greater implications for the biological activities of p53.

Our findings show that Mdm2 sumoylation increases its ubiquitin ligase activities while protecting Mdm2 from self-ubiquitination. This provides an important example of the contribution of SUMO-1 modification in the maintenance of intrinsic E3 ubiquitin ligase activity.

### Example 2

### Identification of a Ubc9 Binding Site on Mdm2

### Materials and Methods

Expression Vectors.

Vectors described in Example I were used in the present example. The sequence of the Ubc-9 binding domain on Mdm2 (aa 40-59, Mdm2 Ubc9 binding site; mu9bs) in wt or in a scrambled orientation (mu9sb) was cloned in frame between EcoRI and XhoI restriction sites into a pcDNA3 that carries the penetration sequence, followed by the sequence encoding the HA-tag. All Mdm2 mutants were cloned in pBluescipt KS. Mutant Ubc9C93S was a gift from Olli Jänne.

Cells.

Cells described in Example I were used in the present example. To monitor non-covalent protein-complexes, cells were lysed as described previously (Buschmann et al., 2000). Western Blot analysis were carried out as described previously. To assure proper separation of small peptides Trycine Gels were used according to Standard Protocols.

Antibodies.

Antibodies described in Example I were used in the present example. Polyclonal antibodies against Sumo1 were produced in rabbits and affinity purified on HA-Sumo columns. 2A10 monoclonal antibodies (Oncogene) were used for Mdm2 analysis. Monoclonal antibodies against Ubc9 were purchased (Transduction Laboratories, Kentucky).

In vitro Conjugation of SUMO-1.

For competition with in vitro sumoylation of Mdm2, a peptide corresponding to the Ubc-9 binding region on Mdm2 (amino acids 40-59) or a non-specific peptide amino acid sequence was added as indicated in Results.

In vivo Sumoylation/Ubiquitination.

Methods described in Example I were followed.

Peptide Conjugation to Amino Link Column

Immunobilization of peptides to an AminoLink® column (Pierce Biochemical) was carried out exactly according to the manufacturer's protocol. In brief, column was equilibrated with coupeling buffer and 1 column volume of solution containing 1 mg of peptide was incubated with gel for 6 h. Later column was drained and washed with coupeling buffer. Coupeling buffer was replaced by quenching buffer containing a reducing solution to block remaining active sites for 30 min. Final washing steps were carried out with washing buffer. Column was stored in storage buffer at 4° C.

UV-treatment Ultraviolet

(UV) irradiation was administered as previously described (Buschmann et al, 2000). Briefly, cells were subjected to UV-C irradiation (254 nm) in calibrated areas within the tissue culture hood. Medium that was removed prior to irradiation was returned to minimize serum-induced changes.

### Results

Ubc9 Binding is Mapped to the Mdm2 Amino-terminal Domain.

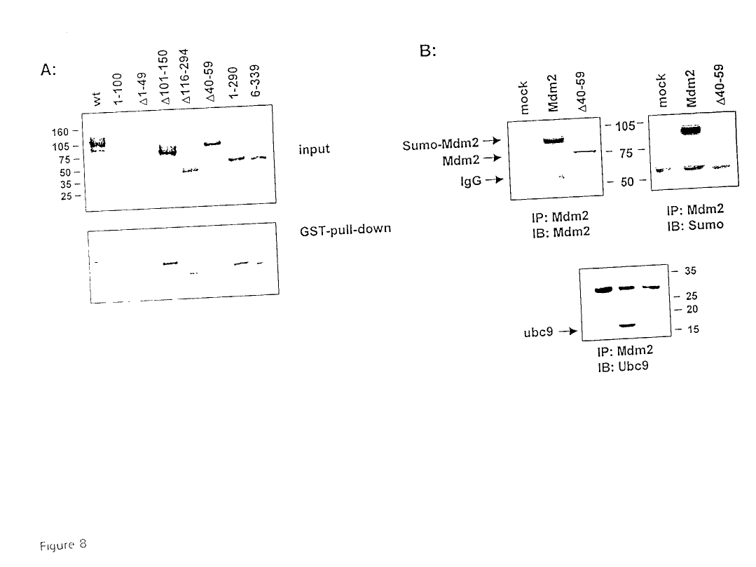

To map the Ubc9 binding site on Mdm2, we incubated a series of ³⁵S labeled-Mdm2 deletion mutants with GST-Ubc9. These GST pull down assays enabled us to determine which of the Mdm2 forms lost the ability to associate with Ubc-9. As shown in FIG. 8A, Mdm2 from which aa 40-59 had been deleted was the only construct that was no longer capable of associating with Ubc-9. This finding suggests that Ubc-9 binding may require aa 40-59 of the amino terminal domain of Mdm2. Forced expression of Mdm2 that lacks aa 40-59 confirmed that this protein is no longer associated with Ubc9 (FIG. 8B). Furthermore Mdm2D40-59 migrates as a 75 kDa protein, which represents the non-sumoylated from of Mdm2 (FIG. 8B). This data confirms that amino acids 40-59 of Mdm2 are required for Ubc9 association.

Ubc9 Binds to a Peptide Corresponding to Amino Acids 40-59from Mdm2.

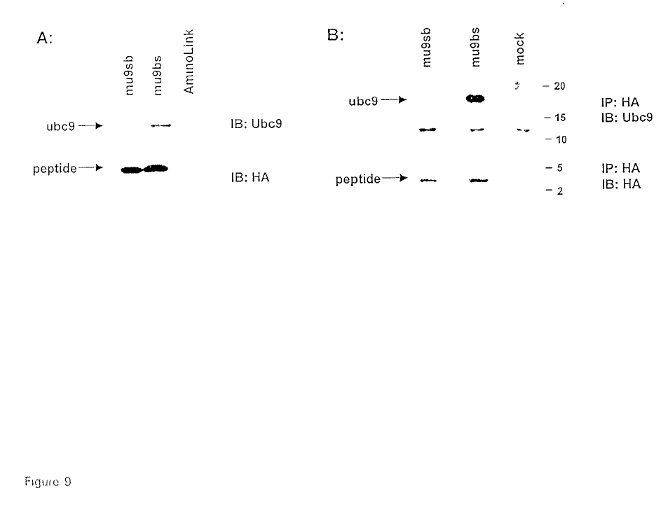

To further assess the role of the Ubc-9 binding domain in Mdm2 sumoylation, a peptide that corresponds to aa 40-59 of Mdm2 was synthesized. We first tested whether the peptide is capable of associating with Ubc9. We coupled either the wt peptide (mu9bs=mdm2 Ubc9 binding site) or a scrambled peptide (mu9sb) to an AminoLink® colunm. Beads coupled to the peptides were incubated with cell lysates and bound proteins were analyzed in Western blots using antibodies to Ubc9. The wt but not mutant (scrambled) peptide captured Ubc9 (FIG. 9A).

To further confirm the ability of the peptide corresponding to mu9bs to associate with Ubc9 we carried out in vivo reactions. We cloned the 19 aa peptide into an HA-tagged mammalian expression vector that also carried the penetratin peptide in frame, assuring its localization into the nucleus. This expression vector was found useful for the expression of short peptides in mammalian cells (Bushmann et al., Mol. Cell. Biol. 21:2743-2754, 2001; Fuschs et al, Oncogene 17:2542-2547, 1998). Immunoprecipitation of the peptide from cells (with antibodies to HA-tag) followed by immunoblot analysis with antibodies to Ubc9 revealed that the mu9bs peptide, but not its scrambled counterpart, associated with Ubc9 (FIG. 9B). These data establish that the region corresponding to aa 40-59 of Mdm2 serves as the Ubc9 binding site.

A Peptide Corresponding to the Ubc9 Binding Site on Mdm2 Attenuates Mdm2 Sumoylation in vitro and in vivo.

To test the effect of the mu9bs peptide on Mdm2 sumoylation, we first determined the kinetics of this reaction. Efficient sumoylation was observed as early as

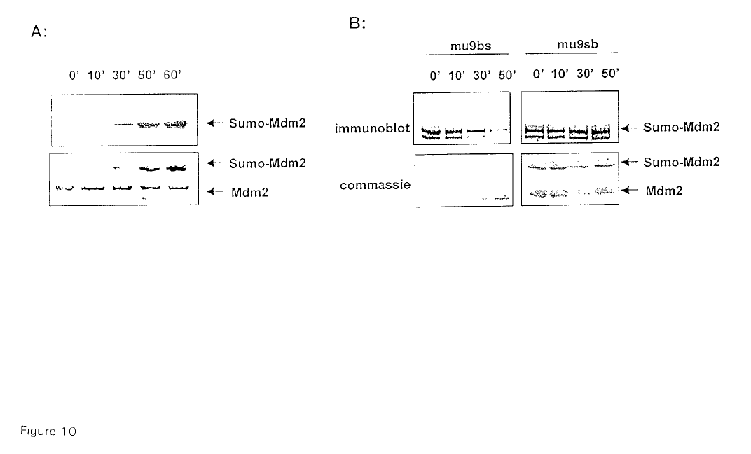

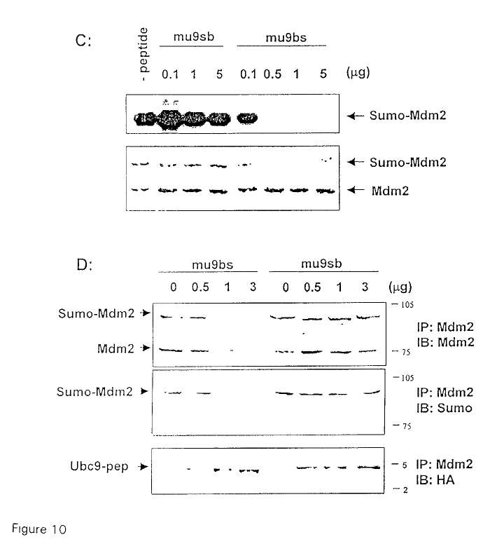

30 min after initiation of the in vitro sumoylation reaction, and it reached maximal levels within 50-60 min (FIG. 1 OA). Addition of the wt mu9bs peptide for different periods of time during the in vitro sumoylation reaction was sufficient to attenuate Mdm2 sumoylation in a manner that coincided with the length of the incubation (FIG. 10B); 0′ reflects no addition of peptide and thus maximal degree of sumoylation; 10′ reflects the degree of sumoylation when the peptide was added for 10 min of the reaction; and 50′ represents the reaction in which the peptide was added for 50 (out of 60′) of the reaction). Similarly, addition of increasing concentrations of mu9bs to the in vitro sumoylation reaction led to a dose-dependent decrease of Mdm2 sumoylation (FIG. 10C). These observations suggest that the presence of the peptide that corresponds to the Ubc9 association site on Mdm2 is sufficient to attenuate Mdm2 sumoylation in vitro.

Important confirmation of the effect of the Mdm2-driven peptide on Mdm2 sumoylation comes from in vivo studies. Co-expression of the mu9bs peptide with Mdm2 in p53/Mdm2 double null cells led to a dose-dependent decrease in the degree of Mdm2 sumoylation in vivo, whereas the mu9sb peptide had no effect (FIG. 10D). Together, these observations identify Mdm2 domain that is required for Ubc9 binding and consequent sumoylation of Mdm2.

Expression of Corresponding to Ubc9 Binding Site Peptide does not Affect the Association of Mdm2 with p53.

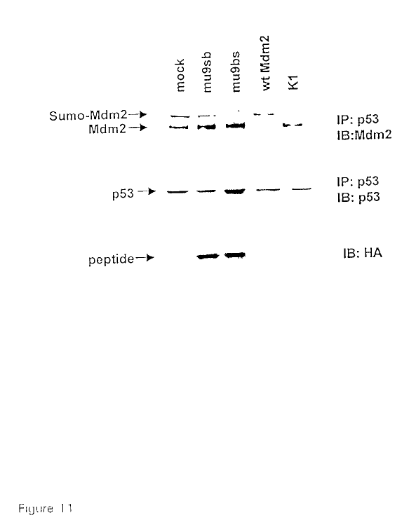

Given the proximity of p53 association on the Mdm2 and Ubc9 binding domains, we tested whether inhibition of Mdm2 sumoylation by over expression of the mu9bs peptide would also affect degree of Mdm2 association with p53. Therefore, we transfected human fibroblasts with mu9bs or mu9sb peptides. Immunoprecipitation of p53 was followed by Western blot analysis to determine the type and level of Mdm2 bound to p53 under each of these conditions. As revealed in FIG. 11, both the 75- and the 90 kDa forms of Mdm2, reflecting the non- and the sumoylated forms, respectively, were found in complex with p53. Whereas expression of the control peptide mu9sb did not affect the association with either form of Mdm2, cells expressing the mu9bs exhibit p53 association only with the non-sumoylated form of Mdm2 (FIG. 11). Forced expression of wt Mdm2 resulted in an association of p90 with p53, whereas expression of the K1 form (a mutant form of Mdm2 on aa 446 which can not be ubiquitinated or sumoylated; (Buschmann et al., Cell 101:753-762, 2000) revealed an association of the non-sumoylated form with p53 (FIG. 11). Effect of mu9bs expression on the form of Mdm2 associated with p53 is expected to also affect level of p53 in the cell. Indeed, overall levels of p53 expression coincided with the type of peptide expressed; mu9bs expression led to increased level of p53, in accordance with decreased sumoylation of Mdm2, which mediates p53 degradation. These data suggest that Ubc9 binding does not affect Mdm2 association with p53, but rather, the form of Mdm2 expressed and associated with p53.

Mutant Ubc9, Which no Longer Conjugates SUMO-1, Decreases Mdm2 Sumoylation and p53 Ubiquitination.

A specific mutation of conserved cysteine (C93S) was previously shown to abrogate Ubc9 ability to conjugate SUMO-1 to androgen receptor while increasing receptor-mediated transcriptional activities (Poukka et al., J. Biol. Chem. 274:19441-19446, 1999). To determine whether Ubc9 association with Mdm2 may affect its ability to target p53 ubiquitination, tested the effect of this mutant form of Ubc9. Addition of Ubc9C93S to in vitro sumoylation reaction of Mdm2 revealed that it failed to cause Mdm2 sumoylation, and was capable of decreasing wt-Ubc-9-mediated Mdm2 sumoylation (FIG. 12A). Corresponding to decreased Mdm2 sumoylation, an increase in self ubiquitination was observed (FIG. 12A). In vivo analysis was carried out via transfection of wt or mutant Ubc9 together with HA-Ub into normal human fibroblasts. Whereas expression of wt Ubc9 increased the level of Mdm2 sumoylation in these cells, the expression of mutant Ubc9C93S resulted in decreased Mdm2 Sumoylation (FIG. 12B). Dose-dependent increases of transfected mutant Ubc9C93 S revealed a corresponding decrease in the level of p53 ubiquitination (FIG. 12B). Similarly, co-expression of K1 mutant which elicits efficient ubiquitination of p53, did not alter its effect on p53 ubiquitination in vivo, suggesting that Ubc9 binding is not required for K1 to ubiquinate and degrade p53 (data not shown). These observation suggest Ubc9 binding to Mdm2, without conjugation of Sumo (i.e. Ubc9 binding per se), are not sufficient to increase Mdm2 ability to target p53 ubiquitination; but rather increases Mdm2 self-ubiquitination and decreases p53 ubiquitination.

Ubc9 Binding to Mdm2 is Decreased After UV-irradiation.

Since UV-irradiation has been shown to reduce the degree of Mdm2 sumoylation we monitored possible changes in the association of Ubc9 with Mdm2 in UV-treated cells. UV-irradiation of NIH3T3 cells that were co-transfected with Mdm2 and Sumo revealed a time-dependent decrease in the binding of Ubc9 to Mdm2, which coincided with decreased Mdm2 sumoylation (data not shown). This observation demonstrates that Ubc9 binding to Mdm2 is under a dynamic regulation and affected by changes elicited upon stress and DNA damage, as shown here for UV-irradiation. Our finding further establishes the requirement for Ubc9 binding to enable sumoylation of Mdm2.

### Discussion

Conjugation of Sumo-1 to Mdm2 is a key event underlying Mdm2's ability to elicit efficient degradation of p53 (Buschmann et al., Cell 101:753-762, 2000). Increased Mdm2 E3 ligase activity towards p53 is explained in light of attenuated Mdm2 self-ubiquitination, since sumoylation of Mdm2 displaces its primary ubiquitin conjugation site. The biological importance of Mdm2 sumoylation requires a better understanding of the regulation of Mdm2 sumoylation. Central to the covalent attachment of Sumo-1 to Mdm2 is the conjugating enzyme Ubc9. Here we identify the Ubc9 binding domain on Mdm2, which is required for Mdm2 sumoylation and concomitant Mdm2 E3 ligase activity towards p53. Mdm2, with its Ubc9 association domain deleted, is no longer capable of either association with Ubc9 or of sumoylation. Further, expression of the peptide that corresponds to this region efficiently inhibited Mdm2 sumoylation both in vitro and in vivo. This region is sufficient for Mdm2 association as revealed by in vitro and in vivo data.

The proximity of the Ubc9 binding site on Mdm2 to that required for association with p53 led us to explore possible effects on Mdm2 association with p53; we found that the Ubc9 association does not affect the Mdm2-p53 association but rather the form (sumoylated or non-sumoylated) of Mdm2 that bound to p53. Importantly, changes in the form of Mdm2 that bound to p53 were reflected in the relative levels of p53 expression. Inhibition of Ubc9 binding led to increased levels of p53.

Interestingly, a recent study by Matunis and colleagues, based on the analysis of 11 proteins that were reported to undergo sumoylation, identified that the LLKXE motif (SEQ ID NO: 2) serves as the target site for Ubc9 binding; where X can be any amino acid or may be deleted. A similar sequence (LLKS; SEQ ID NO: 3) is found on aa 37-40 of Mdm2, proximal to aa 40-59 which was characterized in the present studies. Deletion mutant of Mdm2 that encompass aa 1-49 revealed noticeable binding to Ubc9, suggesting that the actual domain engaged in binding of Mdm2 is indeed within aa 40-59.

Association of Ubc9 may affect protein function, even if sumoylation does not take place. The C93S substitution of Ubc9 prevents Sumo-1 conjugation by abrogating the formation of a thiolester bond between Sumo-1 and Ubc9. Expression of C93S form was previously found to elicit stimulation of androgen receptor-dependent transactivation similar to wt Ubc9. This finding implies that Ubc9 association per se may be sufficient for changes in protein conformation and fimction (Poukka et al., J. Biol. Chem. 274:19441-19446 1999). Along those lines, we tested whether the ability of an Mdm2 mutant at aa 446 (K1) to elicit higher E3 ligase activity towards p53 may be attributed to Ubc9 association, since this mutant can no longer be sumoylated. Our data suggest that K1 elicits its targeting of p53 ubiquitination independent of Ubc9, since the mutant form of Ubc9 did not affect K1 activities. Possible changes in conformation of the Mdm2 RING, which are expected to take place due to the K446 mutation, may contribute to its elevated E3 ligase activities, in addition to increased stability of this mutant.

Support for the dynamic regulation of Ubc9's association with Mdm2 comes from the observation that UV-irradiation decreases binding of Ubc-9 while increasing the association of p19^{ARF}. It is likely that UV-irradiation decreases the association of Ubc-9 as a result of phosphorylation of Mdm2 by stress kinases, including ATM (Maya et al., in press) which are expected to elicit conformational changes in Mdm2, resulting an altered subset of its associated proteins. The distant location of Ubc-9 binding (aa 40-59) from that of Mdm2 sumoylation (aa 446) further supports the importance of Mdm2 conformation in relations to its ability to undergo sumoylation. Overall, the present study adds important new information regarding the association of Mdm2 with Ubc9, which is prerequisite for Mdm2 sumoylation. Our data provide evidence for the existence of dynamic regulation of ubc9 association, which is altered upon UV-treatment. We further demonstrate, for the first time, that Ubc9 association is distant from the site of SUMO-1 conjugation, suggesting that the regulation of Mdm2 sumoylation is highly dependent on protein conformation.

### Example III

### Mechanism of p14^{ARF}

### Materials and Methods

Expression Vectors.

Vectors described in Example I were used. p53wt and p21-luc expression vectors were previously described (Fuchs et al, Oncogene 17:2543-2547, 1998). SUMO-1 expression vector was described previously (Rodriquez et al., EMBO J. 18:6455-6461, 1999). p19, p19^{Δ62} and p₁₉^{Δ1-14, Δ26-37} expression vectors were provided by Drs. C. J. Sherr and M. F. Roussel.

Cells.

Non-denaturing immunoprecipitation and luciferase assay p53/Mdm2 double null cells (Fuch et al., Oncogene 17:2543-2547, 1998), EJ, NIH3T3 mouse fibroblasts and TIG human fibroblasts were maintained in DMEM (Gibco) supplemented with 10% heat-inactivated fetal bovine serum and antibiotics in 5% CO Transfections were performed by lipofection (LipofectaminPlus, GIBCO). Medium was changed 5 h after transfection and cells were harvested 36 h later. To monitor non-covalent protein-complexes, cells were lysed and western blot analysis was performed as described previously (Buschmann et al., Cell 101, 753-762, 2000). Luciferase activity was determined using the Promega luciferase assay system (Promega, Madison, Wis.). Luciferase activities were normalized on the basis of β-galactosidase levels in transfected cells.

Antibodies.

Monoclonal antibodies against HA-epitope were purchased (HA11, BabCo). Monoclonal antibodies against SUMO-1 were purchased (GMP-1; Zymed) and polyclonal antibodies to bacterial SUMO-1 were raised in rabbits followed by affinity purification on a Amino Link SUMO-1 column. Monoclonal antibodies 2A10 (Oncogene) were used for Mdm2 analysis. p53 detection was carried out using monoclonal pAb421 antibodies (Oncogene). Antibodies against p19^{ARF} were a gift of Drs. C. J. Sherr and M. F. Roussel.

In vitro Conjugation of Sumo-1.

Glutathione beads (SIGMA) were used to purify GST-Mdm2 as previously described (Fang et al., J Biol Chem 275:8945-51, 2000). Bacterially expressed purified Mdm2 proteins (1-2 μg) were incubated with ³⁵P-Labeled SUMO-1, Hela cell-purified Aos1/Uba2 (kind gift of C. G. Lee) (15 ng) and bacterially expressed Ubc9 (0.5 μg) for 60 min at 37° C. in conjugation buffer (20 mM Hepes, pH 7.4, 5 mM MgCl₂, 2 mM ATP, 10 mM creatine phosphate, 1 unit creatine phosphokinase) in the presence or absence of cold in vitro-translated or bacculovirus-expressed p19 forms. Bead-bound complexes were washed (3×TBS/0.5% Triton-X-100) before being denatured for 5 min at 95° C. in 3× Sample Buffer. Proteins were separated on 8% SDS-PAGE. Gel was stained with Coomassie blue, dried and exposed to X-ray film (Xomat, Kodak).

In vivo Sumoylation/Ubiquitination.

Cells were transfected with Flag-tagged indicated Mdm2 plasmids, the pertinent p19 constructs, p53wt, and cDNAs encoding either HA-tagged SUMO-1 or HA-tagged Ubiquitin to analyze sumoylation or ubiquitination, respectively, as previously described (Buschmann et al., Cell 101, 753-762, 2000).

Pulse Chase Halflife Measurement.

Normal human fibroblasts (NHF) were transfected with wt or S218A forms of Mdm2 expression vectors (4 μg). ³⁵S-methionine (1 mCi) was added to the cell cultures 24 h after transfection for 5 h (pulse) followed by chase (2 mM cold methionine) for the indicated time points. Protein extracts (1 mg) were used for immunopurification of Mdm2 using mixture of monoclonal antibodies (2A-10/IF2). Immunoprecipated Mdm2 was washed, separated on 10% SDS-PAGE, and analyzed by autoradiography.

### Results

p19^{ARF} Attenuates Mdm2 Sumoylation.

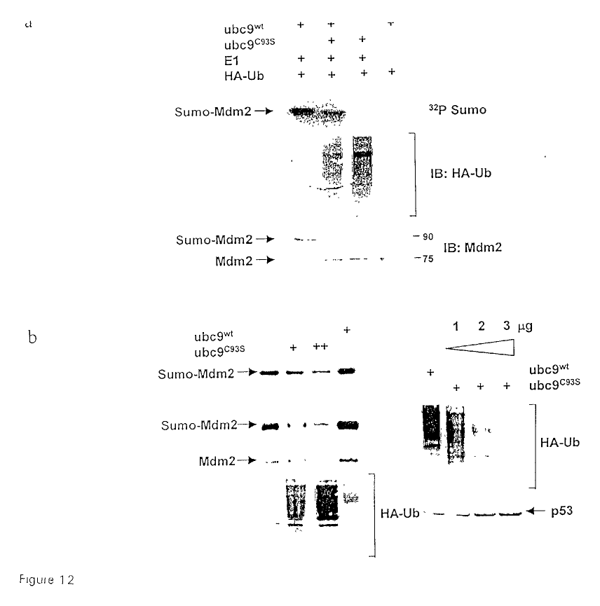

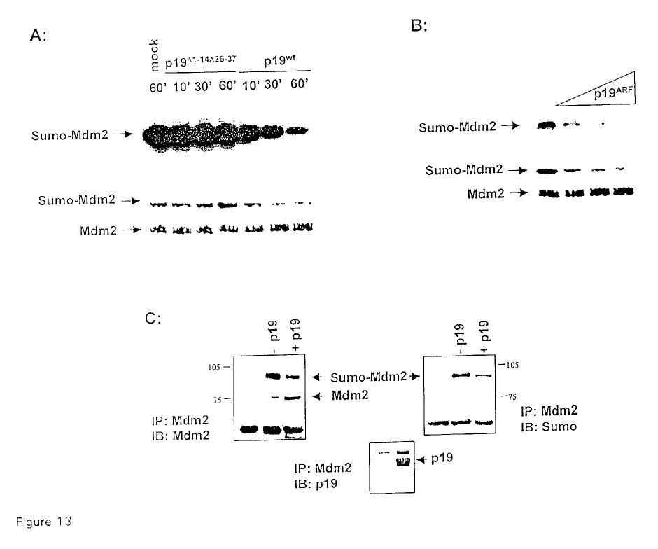

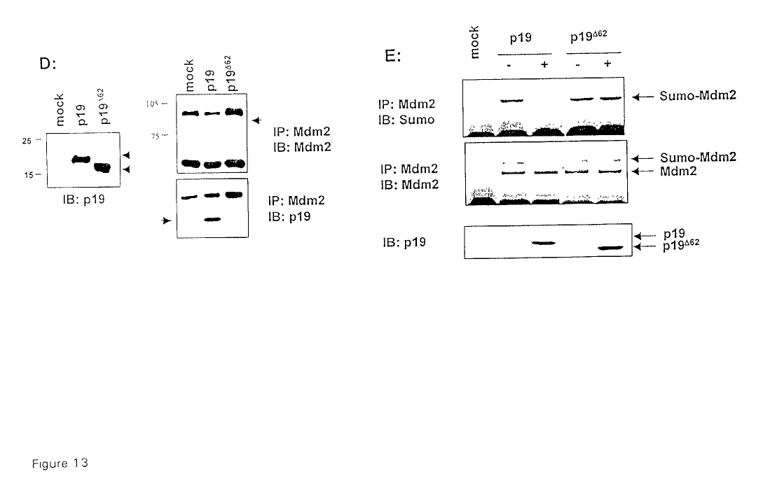

In vitro and in vivo assays were performed to assess whether association of p19 with Mdm2 affects Mdm2 sumoylation. Addition of in vitro-translated wt, but not mutant p19^{ARF} lacking the N-terminal residues required for its binding to Mdm2 (p19^{Δ1-14,26-37}), led to efficient inhibition of GST-Mdm2 sumoylation in vitro (FIG. 13A). Similarly, in vitro sumoylation of Mdm2 was inhibited in a dose-dependent manner by p19^{ARF} purified from insect cells (FIG. 13B). Based on amount of p19^{ARF} required to mediate 50% inhibition of Mdm2 Sumoylation we estimate that an equal molar ratio between p19-Mdm2 is required for such inhibition. To confirm the in vitro findings, we examined the effect of p19^{ARF} on Mdm2 sumoylation in vivo. Co-expression of p19^{ARF} and Mdm2 in Mdm2/p53-/-cells reduced the degree of Mdm2 sumoylation and increased the fraction of non-sumoylated Mdm2 (FIG. 13C). These results suggest that expression of p19^{ARF} decreases conjugation of SUMO-1 to Mdm2 in vitro and in vivo.

To further elucidate the ability of p19^{ARF} to attenuate Mdm2 sumoylation, we used the EJ mouse fibroblast cell line, which lacks p19^{ARF} expression. Forced expression of Mdm2 and wt or mutant (p₁₉^{Δ62}, which lacks the domain required for association with Mdm2) forms of p19^{ARF} identified only the wt form in complex with Mdm2 (FIG. 13D). Co-expression of wt p19^{ARF} with Mdm2 resulted in inhibition of Mdm2 sumoylation, which was not observed upon co-expression of the mutant p19^{Δ62} form (FIG. 13E). These results establish that p19^{ARF} efficiently attenuates the conjugation of SUMO-1 to Mdm2 in vitro and in vivo. Our data also demonstrate association of 19^{ARF} with Mdm2 is required to inhibit Mdm2 sumoylation.

p19^{ARF} Induces Mdm2 Self-ubiquitination.

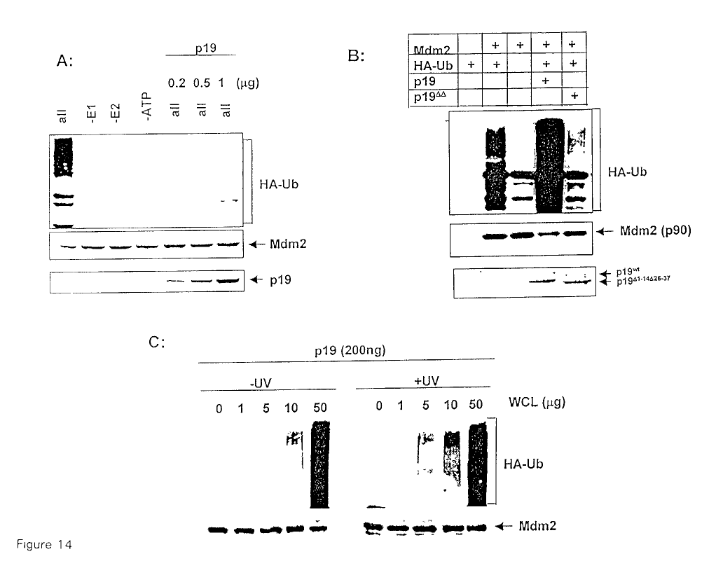

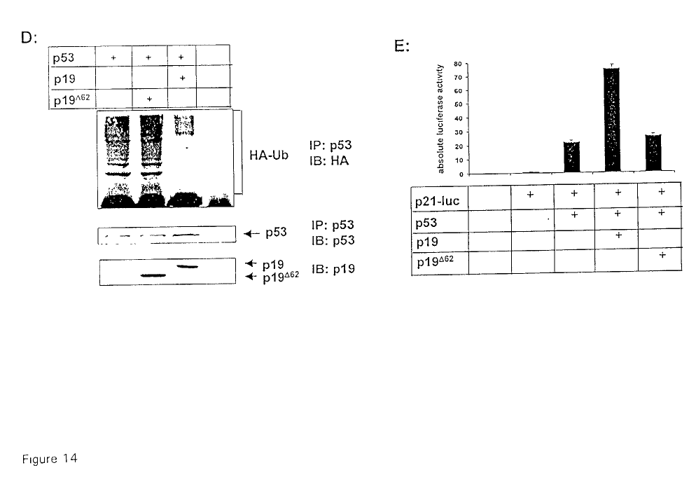

SUMO-1 modification of Mdm2 takes place on the same residue required for Mdm2 ubiquitination (Buschmann et al., Cell 101:753-762, 2000). We, therefore, determined whether reduced Mdm2 sumoylation due to p19^{ARF} expression alters the degree of Mdm2 self- (auto)-ubiquitination. Addition of bacculovirus-purified p19^{ARF} to bacterially expressed and purified GST-Mdm2 led to efficient inhibition of Mdm2 ubiquitination in vitro (FIG. 14A). Although this finding is in accordance with Honda and Yasuda's results (Honda and Yasuda, Oncogene 19:1473-1476, 2000), decreased sumoylation (FIG. 13) was expected to result in increased self-ubiquitination unless the association of p19^{ARF} altered the conformation of the RING domain required for both ubiquitin and SUMO-1 conjugation. In contrast to the effect of p19^{ARF} on Mdm2 ubiquitination in vitro, in vivo ubiquitination assays revealed that Mdm2 ubiquitination was no longer inhibited, but rather increased, upon p19^{ARF} expression. Forced expression of Mdm2 and p19^{ARF} in EJ p19 null cells caused a noticeable increase in the degree of Mdm2 ubiquitination (FIG. 14B). This remarkable difference led us to explore the nature of the opposing effects of p19^{ARF} on Mdm2 ubiquitination in vitro and in vivo. Of interest, forced expression of mutant p19^{Δ62} decreased the degree of Mdm2 self-ubiquitination (FIG. 14D). These finding suggest that Mdm2 may be subjected to in vivo modification which alters p19 effects. To partially mimic the in vivo conditions we tested the effect of p19^{ARF} on ubiquitination of Mdm2 in the presence of protein extracts prepared from cells prior to, and after, UV-treatment. Addition of protein extracts to the in vitro ubiquitination reaction of Mdm2 attenuated, in a dose-dependent manner, the ability of p19^{ARF} to inhibit Mdm2 self-ubiquitination (FIG. 14C). Proteins prepared after UV-treatment were more potent in attenuating p19^{ARF}-mediated inhibition of Mdm2 self-ubiquitination when compared with those prepared from non-treated cells (compare 5 and 10 mg lanes in FIG. 14D). These observations suggest that incubation of Mdm2 with protein extracts led either to post-translational modification(s) of Mdm2 or to the association of other cellular protein(s) with Mdm2 which impede the ability of p19^{ARF} to inhibit Mdm2-self ubiquitination. Taken together, these in vivo and in vitro observations indicate that p19^{ARF} inhibits Mdm2 sumoylation and increases Mdm2 self-ubiquitination in vivo. Concomitantly with an increase in Mdm2 ubiquitination by p19^{ARF}, p19^{ARF} but not p₁₉^{Δ62} elicited a noticeable decrease in the degree of p53 ubiquitination, which coincided with elevated levels of p53 (FIG. 14E). A corresponding increase was also observed in p53 transcriptional activity when EJ cells were transfected with p53, p19^{ARF} and a luciferase construct controlled by p21 promoter sequences that contained p53 consensus binding sites (FIG. 14E). These findings establish that the ability of 19^{ARF} to attenuate Mdm2 sumoylation in mouse fibroblasts results in increased Mdm2 ubiquitination and in a concomitant increase in p53 stability and activity.

p19^{ARF}-mediated Increase of Mdm2 Self-ubiquitination Requires Mdm2 Phosphorylation by MAPK/p38.

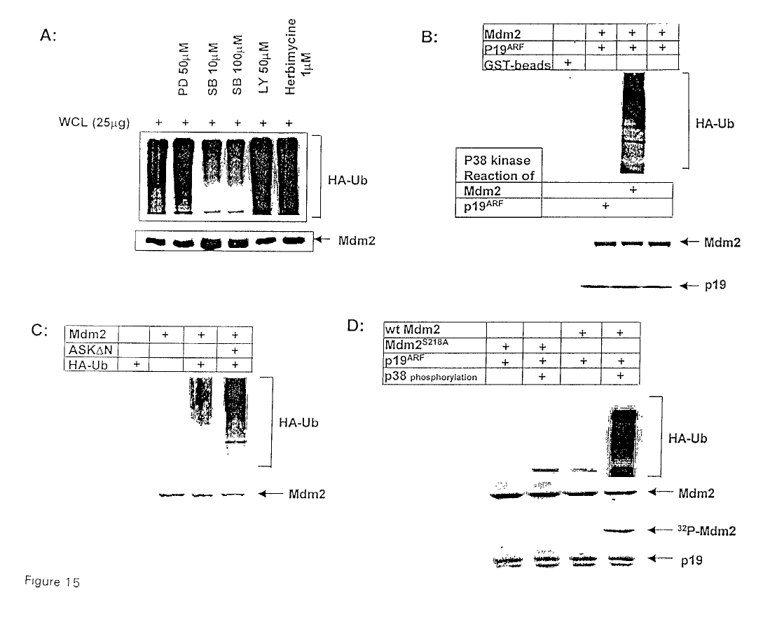

We then tested the possible role of post-translational modification by stress kinases, since the degree of self-ubiquitination was greater in extracts of UV-treated cells (FIG. 14C). To assess the possible contribution of stress kinases to p19^{ARF}-mediated Mdm2 ubiquitination, we treated cells that had been transfected with p19^{ARF}, Mdm2 and HA-Ubiquitin with the pharmacological inhibitors of p38, ERK, JAK or PI3K. Of the different inhibitors, only SB203580, the inhibitor of MAPK/p38 was capable of attenuating p19^{ARF}-mediated Mdm2 ubiquitination (FIG. 15A). This observation suggests that p38 phosphorylation may be required for p19^{ARF}-induced Mdm2 self-ubiquitination.

To determine whether p38 phosphorylation is required on p19^{ARF} or on Mdm2 to enable Mdm2 self-ubiquitination, we carried out in vitro phosphorylation of Mdm2 or p19^{ARF} before performing the in vitro ubiquitination reaction. Subjecting p19^{ARF} to phosphorylation by p38 did not alter its ability to inhibit Mdm2 ubiquitination in vitro. Comparatively, phosphorylation of Mdm2 by p38 resulted in efficient ubiquitination of Mdm2 upon addition of p19^{ARF} (FIG. 15B). These results suggest that phosphorylation of Mdm2 by p38 is required for p19^{ARF}-mediated Mdm2 ubiquitination in vitro.

Further confirmation for this observation comes from in vivo studies. Immunoprecipitation of Mdm2 from cells that express the constitutively active form of p38 upstream kinase (ASKΔN) followed by the in vitro ubiquitination assay revealed marked increase in the ubiquitination of Mdm2 when compared with cells that do not express the active p38 kinase (FIG. 15C). Search for putative sites for p38 phosphorylation on Mdm2 identified one site that consists the classic motif (SP/TP) for MAPK phosphorylation at aa 218. Mdm2 mutated at 218 (S→A) was no longer phosphorylated by p38 nor was it able to undergo p19^{ARF}-mediated ubiquitination upon p38 phosphorylation (FIG. 15D). This finding directly establish the role of Ser 218 and p38 in confering p19^{ARF}'s-ability to induce Mdm2 self-ubiquitination.

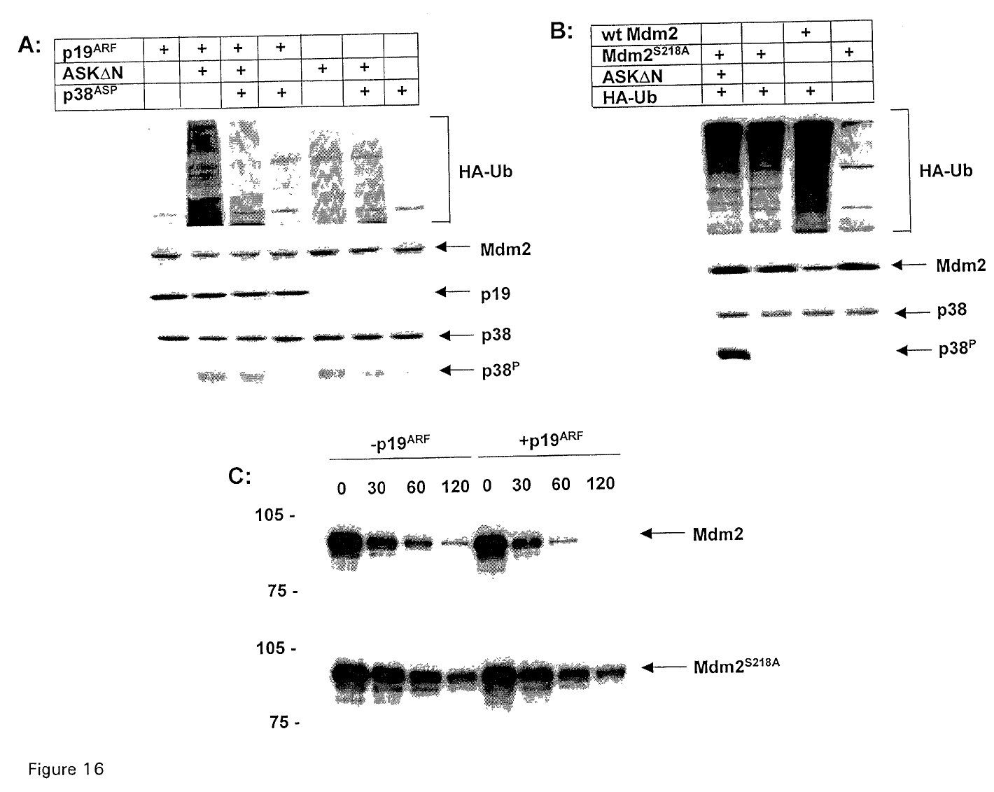

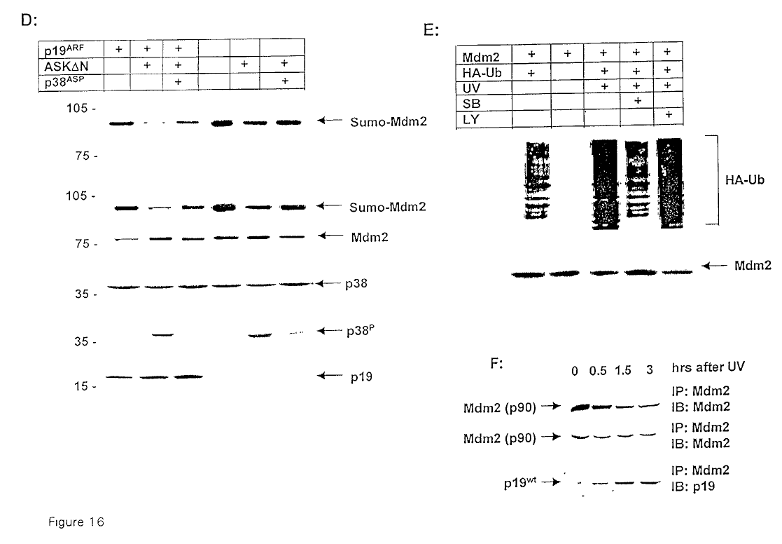

To further elucidate the role of p38 in Mdm2 self-ubiquitination, we tested the effect of p38 on Mdm2 ubiquitination in vivo. In vivo ubiquitination assays revealed a noticeable increase in the ubiquitination of Mdm2 in cells that express the constitutively active form of p38. Co-expression of the dominant negative form of p38 (p38^{ASP}) efficiently attenuated the effect of the upstream kinase ASKΔN (FIG. 16A). These findings provide additional support for the role of p38 in conferring Mdm2 susceptible for ubiquitination. The effect of p38 on Mdm2 ubiquitination was substantially greater in cells that were transfected with p19^{ARF}, when compared to those that lack p19^{ARF} (FIG. 16A). Forced expression of Mdm2^{S218A} revealed a basal degree of ubiquitination which was not affected upon expression of the p38 kinase ASKΔN (compare lanes 1 and 2 FIG. 16B). The level of Mdm2^{S218A} ubiquitination was lower than that seen with the wt form of Mdm2 (compare lanes 2 and 3 in FIG. 1B). These observations are in line with data shown in FIG. 16e, where p38^{ASP} expression failed to completely abolish level of Mdm2 ubiquitination. These findings suggest that additional modifications, which are independent of the p38 kinase and the 218 site take place under normal growth conditions and confer basal degree of Mdm2 ubiquitination. These findings further support the conclusion that the ability of p19^{ARF} to elicit Mdm2 ubiquitination is dependent upon p38 phosphorylation and suggests that p19^{ARF} augments basal ubiquitination of Mdm2 in vivo. Important support for the effects of p19^{ARF} on Mdm2 stability comes from pulse chase experiments. Comparison of wt with S218A mutant form of Mdm2 revealed a substantial increase in the half-life of the p38 phosphomutant (FIG. 16C). The later is in agreement with the finding that this site is required for p19-mediated Mdm2 ubiquitination. Indeed, whereas p19^{ARF} efficiently shorten Mdm2 half-life, it had minimal effects on the S218A mutant (compare upper and lower panels in FIG. 16C). These observations suggest that p19^{ARF} efficiently decrease Mdm2 stability and that the 218 site is required for p19^{ARF}-mediated Mdm2 degradation.

Given the inverse relationship between Mdm2 auto-ubiquitination and its sumoylation, we next determined whether p38 phosphorylation also affects the degree of Mdm2 sumoylation. Forced expression of the ASKΔN construct decreased the degree of Mdm2 sumoylation. Co-expression of the dominant negative form of p38 attenuated the effect of ASKΔN resulting in increased Mdm2 sumoylation (FIG. 16D). Importantly, the effect of p38 on Mdm2 sumoylation was more pronounced in the presence of p19^{ARF} (compare p19+ and − panels in FIG. 16D). These data suggest that Mdm2 phosphorylation by p38 potentiates p19^{ARF} ability to decrease its sumoylation while increasing its auto-ubiquitination.

Since p38 is among stress kinases that are activated in response to various external stimuli, including UV-irradiation, we have tested the possible contribution of p38 to Mdm2 ubiquitination following UV treatment. UV-irradiation led to a noticeable increase in level of Mdm2 ubiquitination, which was attenuated in cells that were pre-treated with the pharmacological inhibitor of p38, but not by the pharmacological inhibitor of PI3K (FIG. 16E). These data provides a relevant physiological example for the regulation of Mdm2 ubiquitination upon p38 phosphorylation. Increased Mdm2 ubiquitination following UV-treatment coincided with increased amount of p19^{ARF} that was found in complex with Mdm2 (FIG. 16F). These observations are in line with the finding that p19^{ARF} decreases Mdm2 sumoylation while increasing its self-ubiquitination in a phosphorylation dependent manner.

P19^{ARF} Association with—and p38 Phosphorylation of—Mdm2 Decreases Ubc9 Binding.

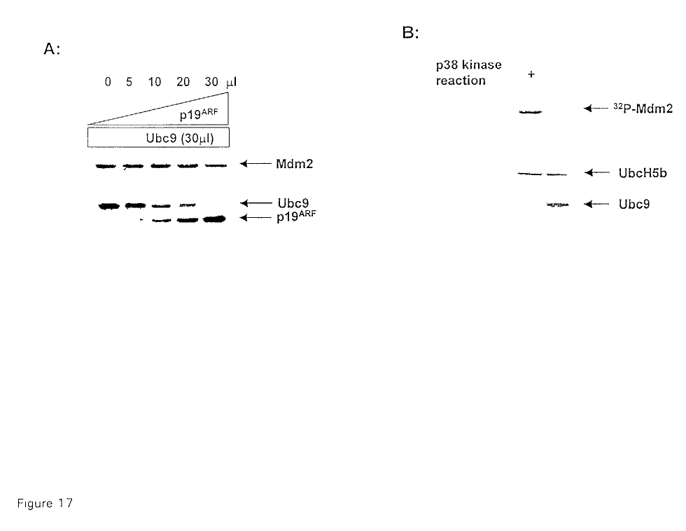

Increased ubiquitination by p19^{ARF} upon p38 phosphorylation concomitant with decreased Mdm2 sumoylation led us to determine possible mechanisms for these changes. A prerequisite for Mdm2 sumoylation is the association of Ubc9, the SUMO conjugating enzyme, with Mdm2, which enables subsequent conjugation of SUMO-1 to Mdm2. Inhibition of Ubc9's association with Mdm2 attenuates Mdm2 sumoylation. Given the effect of p19^{ARF} on Mdm2 sumoylation, we proceeded to determine whether p19^{ARF} may alter Ubc9 binding to Mdm2. To this end we performed an in vitro binding assay, using in vitro translated p19^{ARF} and Ubc-9, as indicated. Incubation of Mdm2 with Ubc-9 in the presence of increasing amounts of p19^{ARF} revealed a dose dependent decrease in Ubc9 binding to Mdm2 (FIG. 17A). That binding of p19^{ARF} efficiently reduces the association of Mdm2 with Ubc9 provide a mechanism underlying p19^{ARF} ability to decrease Mdm2 sumoylation. We next assessed possible changes in Ubc9 binding to Mdm2 upon its phosphorylation by p38. In vitro phosphorylation of Mdm2 by p38 led to a marked decrease in Ubc9 binding to Mdm2 (FIG. 17B). Unlike changes in Ubc9 association, only a modest increase was seen in UbcH5b binding to Mdm2 upon its phosphorylation. This observation further explains the nature of decreased Mdm2 sumoylation in response to UV-irradiation, which is a potent activator of p38 kinase.

### Discussion

The ability of p19^{ARF} to attenuate Mdm2 E3 ligase activity towards p53 has been reported, but the mechanisms underlying this change are not completely understood. Here we demonstrate that p19^{ARF} decreases Mdm2 sumoylation, and increases, in a phosphorylation-dependent manner, Mdm2 self-ubiquitination, thereby limiting the amount of Mdm2 available for targeting p53 degradation. Our study provides the mechanisms underlying p19^{ARF} inhibition of Mdm2 sumoylation, namely, via its ability to inhibit the association of Ubc9 with Mdm2. We further demonstrate that p38 is the primary kinase required for p19^{ARF}-ability to increase Mdm2 self ubiquitination while attenuating its sumoylation, an event that takes place in response to stress as demonstrated here for UV-irradiation.

Decreased Mdm2 sumoylation coincides with increased Mdm2 self-ubiquitination, which is expected to result in its efficient degradation. Our results are consistent with the findings of Zhang et al., 1998, who demonstrated that p19^{ARF} promotes Mdm2 degradation and stabilizes p53. We identified Serine 218 of Mdm2 as the phosphoacceptor site for p38 and demonstrated that a S218A mutant no longer enabled p19^{ARF}-mediated Mdm2-self ubiquitination. It is important to note that basal level of Mdm2 ubiquitination exist and is not dependent on either p19^{ARF} or p38 phosphorylation, suggesting that other cellular factors and signaling pathways control basal Mdm2 ubiquitination. Yet, p19^{ARF} elicits a significant increase in the level of Mdm2 ubiquitination, which takes place upon activation of p38 kinases, in response to growth factors, stress, or certain oncogenic stimuli. Indeed, we demonstrate that UV-treatment increases binding of p19^{ARF}, which coincides with increased Mdm2 ubiquitination and decreased Ubc9 association and Mdm2 sumoylation.

The effect of p19^{ARF} on Ubc9 binding to Mdm2 is central to its ability to inhibit Mdm2 sumoylation. The regulation of Mdm2's association with either Ubc9 or p19^{ARF} is expected to be at the level of post-translational modifications, which may alter Mdm2's conformation to favor association with one protein over the other, and consequently determine Mdm2's fate with respect to sumoylation or self-ubiquitination. The latter is further supported in light of the finding that phosphorylation of Mdm2 by p38 attenuates Ubc9 binding and facilitates p19^{ARF}-mediated Mdm2 self-ubiquitination. Given that binding of p19^{ARF} to Mdm2 attenuates its E3 ligase activity (Honda and Yasuda, Oncogene 10: 1473-1476, 2000; Sharp et al., J. Biol. Chem. 274:38189-38196, 1998; Pomerantz et al., Cell 92:713-723, 1998), as opposed to the binding of Ubc9, which acquires greater E3 ligase activity (Buschmann et al., Cell 101:753-762, 2000), this interplay highlights a new layer of regulation of Mdm2 oncogenic activities, which has been implicated in the efficient degradation of p53.

It is important to note that UV irradiation also alters the degree of Ubc9 association with Mdm2 in cells that lack p19^{ARF} expression, although the kinetics of Mdm2 sumoylation and ubiquitination are noticeably slower (data not shown). Changes in the kinetics of Mdm2 ubiquitination and concomitant p53 expression are expected to affect the rate and efficiency of the cellular stress response at the level of growth arrest or apoptosis. These observations suggest that other Mdm2-associated proteins may affect Ubc9 association; such as E2F1, MdmX and p300 (Sharp et al., 2000; Zhu et al., J. Biol. Chem 274:38189-38196, Cell. Growth Differ. 10:829-838, 1999; Dimri et al., Mol. Cell. Biol. 20:273-285, 2000; Martin et al., Nature 375:691-694, 1995; Grossman et al., Mol. Cell. 2:905-415, 1998). That p38 elicited an increase in Mdm2 ubiquitination in the absence of p19^{ARF}, albeit at lower efficiency, further suggests that other cellular proteins may augment p38's ability to target Mdm2 self-ubiquitination while decreasing its association with Ubc9 and subsequent sumoylation. Given that p19^{ARF} also elicits p53-independent cellular regulatory functions along tumor surveillance (Weber et al., Genes Dev. 14:2358-2365, 2000), it is possible that other cellular targets of p19^{ARF} may be subject to regulation similar to that shown here for Mdm2.

The ability of p19^{ARF} to decrease Mdm2 sumoylation, while increasing its self-ubiquitination is likely to be cell type specific. Whereas our studies were carried out in human mouse fibroblasts, forced expression of p19^{ARF} in U2OS cells resulted in elevated expression of Mdm2 (Lohrum et al, Nature Cell Biol. 2:179-181, 2000). The nature of these cell-type differences could be attributed to lack of central regulatory proteins as well as to altered balance of protein kinases/phosphatases, which in certain cases is p53-dependent (Takekawa, et al. EMBOJ. 19:6517-6526, 2000; Lee et al., Prog. Natl. Acad. Sci. 97:8302-8305, 2000).

Association of p₁₉^{ARF} with Mdm2 was shown to block nucleo-cytoplasmic shuttling (Tao and Levine, Proc. Natl. Acad. Sci 96:6937-6941, 1999 ) and to sequester Mdm2 into the nucleolus, thereby preventing negative feedback regulation of p53 by Mdm2 (Weber et al, Nat. Cell. Biol. 1:20-26, 1999). p19^{ARF} binding to Mdm2 was also shown to unmask a cryptic nucleolar localization signal on Mdm2 which coincided with p53-dependent cell cycle arrest in response to stress (Weber et al, Genes Dev. 14:2358-2365, 2000; Lohrum et al., Nature Cell Biol. 2:179-181, 2000). Since nucleolar co-localization may not account for all cellular Mdm2/p19/p53, it is expected that other mechanisms also contribute to the regulation of Mdm2 and concomitantly, p53 stability.

Taken together, the current study points to the regulation of Mdm2 sumoylation and self-ubiquitination by p19^{ARF} and to the role of p38-mediated Mdm2 phosphorylation in this process, which is central to Mdm2's oncogenic activity.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

It is further to be understood that all values are approximate, and are provided for description.

Patents, patent applications, procedures, and publications cited throughout this application are incorporated herein by reference in their entireties.

What is claimed is:

1. A method for modulating Mdm2 activity by altering the level of sumoylation of Mdm2 protein.

2. The method of claim 1, wherein increasing sumoylation of the Mdm2 protein increases Mdm2 stability and Mdm2 E3 ligase activity.

3. The method of claim 1, wherein decreasing sumoylation of Mdm2 decreases Mdm2 stability.

4. The method of claim 3, wherein decreasing sumoylation of Mdm2 comprises increasing ubiquitination of Mdm2.

5. A method of modulating p53 activity, which method comprises modulating sumoylation of Mdm2, wherein the sumoylation of Mdm2 increases ubiquitination of p53 resulting in a decrease in p53 activity, and inhibition of sumoylation of Mdm2 decreases ubiquitination of p53 resulting in an increase in p53 activity.

6. The method of claim 5, which comprises decreasing sumoylation of Mdm2, thereby decreasing ubiquination of p53.

7. The method of claim 6, wherein decreasing sumoylation of Mdm2 comprises increasing ubiquitination of Mdm2.

8. A method of detecting sumoylation of Mdm2, which method comprises detecting the presence of sumoylated Mdm2 isolated from a mixture comprising Mdm2 protein, SUMO-1, Aos1/Uba2, and Ubc9, wherein the mixture is incubated for a time sufficient for sumoylatin of Mdm2 to occur under control conditions.

9. The method of claim 8, wherein the SUMO-1 is radiolabeled.

10. The method of claim 9, wherein the radiolabel is ³⁵S.

11. The method of claim 8, wherein the SUMO-1 is epitope-tagged.

12. The method of claim 9, wherein the SUMO-1 is a hemagluttinin (HA)-tagged SUMO-1.

13. The method of claim 8, wherein the mixture is a cell-free mixture.

14. The method of claim 8, which further comprises culturing cells transfectedwith an expression vector encoding Mdm2 and an expression vector encoding SUMO-1, under conditions that permit expression of sufficient quantities of Mdm2 and SUMO-1 to permit detection of sumoylated Mdm2.

15. The method of claim 14, which further comprises lysing the cells and clarifying the lysate prior to detecting the presence of sumoylated Mdm2.

16. An assay system for identifying a test compound that regulates sumoylation of Mdm2, comprising Mdm2 protein, SUMO-1, Aos1/Uba2, and Ubc9, wherein the level of these components is sufficient to detect Mdm2 sumoylation.

17. The assay system of claim 16, wherein the components are cell free.

18. The assay system of claim 16, comprising cells transfected with an expression vector encoding Mdm2 and an expression vector encoding SUMO-1, wherein the population of transfected cells expresses sufficient levels of Mdm2 and SUMO-1 to detect Mdm2 sumoylation.

19. A method for identifying a test compound that modulates sumoylation of Mdm2, which method comprises detecting a difference in the level of sumoylation of Mdm2 in an assay system of claim 16 contacted with a test compound, wherein a difference in the level of sumoylation of Mdm2 indicates that the test compound selectively modulates sumoylation of Mdm2.

20. The method of claim 19, wherein the assay system is cell free.

21. The method of claim 19, which method comprises detecting a difference in the level of sumoylation of Mdm2 in an assay system comprising cells transfected with an expression vector encoding Mdm2 and an expression vector encoding SUMO-1, wherein the population of transfected cells expresses sufficient levels of Mdm2 and SUMO-1 to detect Mdm2 sumoylation, wherein one population of transfected cells is contacted with a test compound, wherein a difference in the level of sumoylation of Mdm2 in the population of cells contacted with the test compound compared to a population of cells not contacted with the test compound indicates that the test compound modulates sumoylation of Mdm2.

22. A method for detecting the amount of sumoylated Mdm2 in a sample, which method comprises probing for sumoylated Mdm2 in an immunoprecipitate of Mdm2 from a sample.

23. A method of treating a condition of uncontrolled cell growth, which method comprises inhibiting sumoylation of Mdm2.

24. The method of claim 23, wherein the condition of uncontrolled cell growth is a cancer.

25. The method of claim 24, wherein the cancer is associated with a decrease in p53 activity in the cancer cells.

26. The method of claim 24, further comprising treating the cancer with a p53 gene therapy.

27. The method of claim 2, wherein increased sumoylation is produced by binding of Ubc9 to the Ubc9 binding domain on Mdm2.

28. The method of claim 27, wherein the Ubc9 binding domain on Mdm2 comprises amino acids 40-59 on Mdm2.

29. The method of claim 4, wherein increased ubiquitation is produced by p19^{ARF}.

30. The method of claim 29, wherein the method further comprises increasing phosphorylation of Mdm2.

31. The method of claim 30, wherein p38 increases the phosphorylation of Mdm2.


SEQUENCE LISTING (3 sequences)

SEQ ID NO: 1 - protein, 491 aa, Homo sapiens
MCNTNMSVPTDGAVTTSQIPASEQETLVRPKPLLLKLLKSVGAQKDTYTMKEVLFYLGQYIMTKRLYDEKQQHIVYCSNDLLGDLFGVPSFSVKEHRKIYTMIYRNLVVVNQQESSDSGTSVSENRCHLEGGSDQKDLVQELQEEKPSSSHLVSRPSTSSRRRAISETEENSDELSGERQRKRHKSDSISLSFDESLALCVIREICCERSSSSESTGTPSNPDLDAGVSEHSGDWLDQDSVSDQFSVEFEVESLDSEDYSLSEEGQELSDEDDEVYQVTVYQAGESDTDSFEEDPEISLADYWKCTSCNEMNPPLPSHCNRCWALRENWLPEDKGKDKGEISEKAKLENSTQAEEGFDVPDCKKTIVNDSRESCVEENDDKITQASQSQESEDYSQPSTSSSIIYSSQEDVKEFEREETQDKEESVESSLPLNAIEPCVICQGRPKNGCIVHGKTGHLMACFTCAKKLKKRNKPCPVCRQPIQMIVLTYFP

SEQ ID NO: 2 - protein, 5 aa, Artificial Sequence
  Sumoylation motif
LLKXE

SEQ ID NO: 3 - protein, 4 aa, Artificial Sequence
  Mdm2 sequence amino acids 38-41
LLKS